(19) United States
(12) Patent Application Publication
(10) Pub. No.: US 2026/0294286 A1
(43) Pub. Date: Oct. 1, 2026

(54) MENSTRUAL GARMENT ATTACHMENT

(71) Applicant: SENSIBLE PAD, INC., Chapel Hill, NC (US)

(72) Inventors: Nandini KANTHI, Cary, NC (US); Ishita Bafna, Morrisville, NC (US); Jingyao Deng, Glen Allen, VA (US); Vaishnavi Kode, Apex, NC (US); Prisha Rathi, Apex, NC (US); Juliet Wu, Cary, NC (US)

(73) Assignee: SENSIBLE PAD, INC., Chapel Hill, NC (US)

(21) Appl. No.: 19/635,189

(22) Filed: Mar. 31, 2026

Related U.S. Application Data
(60) Provisional application No. 63/780,749, filed on Mar. 31, 2025.

(51) Int. Cl. A61B 5/145; A61B 5/00; A61B 5/1455; A61B 5/1477

(52) U.S. Cl. CPC A61B 5/14507; A61B 5/1455; A61B 5/1477; A61B 5/4318; A61B 5/6804

(57) ABSTRACT

A removable menstrual garment attachment for tracking reproductive health biomarkers in menstrual fluid is provided. The menstrual garment attachment is configured as a self-contained, single-use adhesive diagnostic module applied to the exterior surface of any commercially available menstrual product. The device includes a detection module that may comprise a lateral flow immunoassay, electronic sensors, or both, along with a microfluidic system for fluid channeling and optional volume regulation. A near-field communication (NFC) chip may be included for wireless data transmission to a mobile device application for longitudinal health tracking. Methods for detecting reproductive health biomarkers include collecting menstrual fluid, channeling through a microfluidic system, optionally regulating fluid volume, filtering contaminants, detecting target analytes, and producing a user-readable output.

### CROSS-REFERENCE TO RELATED APPLICATIONS

This application claims priority to U.S. Application No. 63/780,749, filed on Mar. 31, 2025, which is hereby incorporated by reference in its entirety.

### FIELD OF INVENTION

The present disclosure relates to attachable diagnostic devices that can be added to and removed from menstrual garment products (e.g., menstrual pads, panty liners, absorbent underwear, absorbent activewear, absorbent swimwear, and other menstrual absorbent articles).

### BACKGROUND

Reproductive health conditions, including endometriosis and other inflammatory disorders, remain challenging to diagnose due to limitations in existing diagnostic approaches. Menstrual fluid contains biomarkers that may serve as indicators of inflammatory activity within the menstrual environment. However, current diagnostic methods for detecting such biomarkers present various challenges that limit their accessibility and clinical utility.

Existing approaches to menstrual fluid analysis include sensors embedded within absorbent layers of feminine hygiene products. While such designs can collect diagnostic samples, they may present challenges related to sample extraction and device complexity. Other approaches employ electrochemical detection systems for analyzing menstrual fluid biomarkers. However, electrochemical detection requires electronic components and power sources, which can increase device cost and complexity while limiting accessibility in resource-constrained settings.

Some diagnostic approaches utilize remote laboratory analysis of dried blood spots collected from menstrual fluid. While these methods can provide accurate results, they require users to ship samples to laboratories and await results, which delays diagnosis and limits accessibility for populations lacking shipping infrastructure. Additionally, laboratory testing involves processing costs that can reduce affordability. Reusable diagnostic pad designs incorporating lateral flow immunoassay technology have been developed to provide noninvasive, lab-free detection. However, reusable designs can increase product costs and require users to properly sanitize their pads between uses, presenting challenges for individuals who may lack resources for proper cleaning and maintenance.

Existing lateral flow assay approaches for analyzing blood samples have employed differential chromatographic separation techniques to separate hemoglobin from target analytes and reduce detection interference. Other microfluidic approaches have utilized capillary-driven flow control with meniscus -inhibiting transitions to regulate sample flow. Additionally, some assay methods measure enzymatic activity of biomarkers such as MPO using chromogenic substrates. However, enzymatic activity measurements can present standardization challenges in heterogeneous menstrual blood samples. Furthermore, existing microfluidic devices for body fluid collection have employed metering sections for volumetric measurement, but such approaches may not provide precise triggering of analytical processes.

Accordingly, there exists a need for a single-use/removable wellness device that can provide noninvasive, lab-free detection of reproductive health biomarkers using menstrual fluid for longitudinal biomarker tracking.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

The present invention is generally directed to a removable, adhesive-backed diagnostic device configured, and methods of using thereof, to be applied to the exterior surface of any commercially available menstrual product (“menstrual garment attachment”), wherein said attachment independently contain a complete analytical system, including, e.g., fluid channeling, optional volume regulation, and a detection module capable of producing a readable output. As used herein, the terms “diagnostic” and “wellness” are used interchangeably throughout this application, and the menstrual garment attachment of the present invention can provide readings beneficial to both diagnostic and wellness purposes, including longitudinal biomarker tracking for personalized health monitoring.

Unlike collection-only attachments that transfer a menstrual fluid sample to an external laboratory for analysis, the menstrual garment attachment of the present invention performs at least one analytical or detection step within the device itself, producing a user-readable output at the point of use without requiring sample transfer or remote laboratory processing.

In an aspect, a menstrual garment attachment for tracking reproductive health biomarkers in menstrual fluid is provided. The menstrual garment attachment includes a housing configured to be removably attached to a menstrual absorbent article via an adhesive backing. A detection module is disposed within the housing and configured to detect one or more target analytes in the menstrual fluid and produce a user-readable output. A microfluidic system is configured to channel menstrual fluid toward the detection module. A translucent polymer layer is positioned over a first portion of the detection module and exposes a second portion of the detection module.

In some aspects, the detection module comprises a lateral flow immunoassay embedded in a nitrocellulose membrane.

In some aspects, the detection module comprises one or more electrochemical sensors configured to measure an electrical signal correlated with the concentration of one or more target analytes.

In some aspects, the menstrual garment attachment further includes wings extending from opposite sides of the menstrual garment attachment.

In some aspects, the menstrual garment attachment further includes clasps configured to secure the wings.

In some aspects, the translucent polymer layer is bonded to the housing and to the detection module to form a seal.

In some aspects, the lateral flow immunoassay is disposed within a plastic casing.

In some aspects, the translucent polymer layer comprises a hydrophobic, optically transparent, biocompatible polymer.

In some aspects, the hydrophobic polymer comprises one of polyisoprene, silicone, polyethylene, or polypropylene.

In some aspects, the hydrophobic polymer is selected from the group consisting of polyisoprene, silicone, polydimethylsiloxane (PDMS), polyethylene, polypropylene, thermoplastic polyurethane (TPU), cyclic olefin copolymer (COC), and cyclic olefin polymer (COP). Each of these materials provides suitable optical transparency, biocompatibility, and hydrophobic properties for the cover layer application.

In some aspects, the lateral flow immunoassay includes a collection pad positioned at a first end of the lateral flow immunoassay.

In some aspects, the lateral flow immunoassay further includes a sample pad. A capillary conduit connects the collection pad to the sample pad. A buffer is disposed within the capillary conduit.

In some aspects, a fluid-conditioning reagent is disposed within the capillary conduit. The fluid-conditioning reagent may comprise a buffer, a surfactant, a blocking agent, or combinations thereof. The fluid-conditioning reagent conditions the menstrual fluid as it passes through the capillary conduit, adjusting pH, reducing non-specific binding, or otherwise preparing the sample for interaction with detection reagents in subsequent components of the lateral flow immunoassay.

In some aspects, the lateral flow immunoassay further includes a conjugate pad. A results region includes test lines and control lines. The lateral flow immunoassay further includes a wick pad.

In some aspects, the porous membrane of the lateral flow immunoassay comprises nitrocellulose, polyvinylidene fluoride (PVDF), nylon, or combinations thereof. The porous membrane provides a substrate for immobilization of capture reagents and supports capillary flow of the menstrual fluid sample through the detection zone.

In an aspect, a menstrual garment attachment for detecting disease biomarkers in menstrual fluid is provided. The menstrual garment attachment includes a housing configured to be removably attached to a menstrual pad via an adhesive backing. An electronic sensor is disposed within the housing and configured to detect one or more disease biomarkers in menstrual fluid. A near-field communication (NFC) chip is disposed within the housing and operatively coupled to the electronic sensor. The NFC chip is configured to transmit data corresponding to the detected one or more disease biomarkers to a mobile device application.

In some aspects, the results region of the lateral flow immunoassay comprises one or more test lines and one or more control lines arranged to perform a sandwich immunoassay, competitive inhibition assay, or multiplex detection of two or more target analytes via spatially separated test zones. The sandwich immunoassay format is suitable for detecting larger analytes such as proteins and antibodies. The competitive inhibition assay format is suitable for detecting smaller analytes such as hormones and small molecules. The multiplex detection format enables simultaneous detection of multiple biomarkers from a single menstrual fluid sample.

In some aspects, the electronic sensor comprises an electrochemical sensor.

In some aspects, the mobile device application is configured to store data corresponding to the detected one or more disease biomarkers for longitudinal tracking.

In some aspects, the mobile device application is configured to store and aggregate detection data across a plurality of menstrual cycles for longitudinal tracking, establish a within-individual baseline for one or more target analytes, and generate cycle-specific contextual output relative to the individual's prior cycle data. The within-individual baseline accounts for natural variation in biomarker levels between individuals and provides a personalized reference point for interpreting current results. The cycle-specific contextual output enables the user to understand how current biomarker levels compare to their own historical patterns rather than population averages.

In some aspects, the menstrual garment attachment further includes a microfluidic system configured to channel menstrual fluid toward the electronic sensor.

In an aspect, a menstrual garment attachment for detecting disease biomarkers in menstrual fluid is provided. The menstrual garment attachment includes a housing configured to be removably attached to a menstrual pad via an adhesive backing. A lateral flow immunoassay is disposed within the housing. A near-field communication (NFC) chip is disposed within the housing. The NFC chip is configured to transmit data corresponding to results of the lateral flow immunoassay to a mobile device application.

In some aspects, the mobile device application is configured to store data corresponding to the results of the lateral flow immunoassay for longitudinal tracking.

In some aspects, the menstrual garment attachment further includes a microfluidic system configured to channel menstrual fluid toward the lateral flow immunoassay.

In an aspect, a system for tracking reproductive health biomarkers in menstrual fluid is provided. The system includes a menstrual garment attachment comprising a housing configured to be removably attached to a menstrual absorbent article via an adhesive backing, a detection module disposed within the housing and configured to detect one or more target analytes in the menstrual fluid and produce a user-readable output, and a microfluidic system configured to channel menstrual fluid toward the detection module. The system further includes a wireless communication module integrated into the menstrual garment attachment, wherein the wireless communication module comprises a near-field communication (NFC) chip or a Bluetooth low energy (BLE) transmitter. The system further includes a mobile device application configured to receive, display, store, and analyze transmitted results over time for longitudinal trend analysis.

In an aspect, a method for detecting one or more reproductive health biomarkers using menstrual fluid collected within a menstrual garment attachment is provided. The method includes collecting menstrual fluid via a pad-facing inlet of the menstrual garment attachment. The method includes channeling the menstrual fluid through an internal microfluidic system. The method includes optionally regulating fluid volume via a volume-metered membrane gating mechanism configured to allow a defined fluid volume to pass upon reaching a calibrated threshold. The method includes filtering the menstrual fluid to remove cellular debris, tissue fragments, and microbial contaminants. The method includes detecting one or more target analytes via a detection module. The method includes producing a user-readable output.

In some aspects, the visible result is available within a predetermined time period of menstrual fluid contact with the inlet.

In some aspects, the hydrophilic nanoporous lipid-based membrane ruptures at a calibrated volume threshold of sample flow.

In some aspects, the filtering excludes particles above a predetermined particle size.

In some aspects, the detection module includes multi-zone detection configured to detect one or more analytes selected from the group consisting of inflammatory cytokines, reproductive hormones, oncoproteins, enzymes indicative of inflammatory activity, pathogen-associated antigens, and hormone receptor binding proteins.

In some aspects, the visible result includes colorimetric nanoparticles visible through a transparent window embedded in the menstrual garment attachment.

In some aspects, the one or more reproductive health biomarkers are associated with conditions selected from the group of cervical cancer, endometriosis, breast cancer, uterine fibroids, and pelvic inflammation.

In some aspects, the one or more reproductive health biomarkers comprise myeloperoxidase (MPO) as a wellness indicator for inflammatory activity within the menstrual environment.

In some aspects, the microfluidic system includes directional flow barriers. The directional flow barriers include hydrophobic coating to restrict backflow.

In some aspects, the method further includes comparing the visible result to one or more previous results to establish personalized trend analysis.

In some aspects, the method further includes normalizing a detected analyte signal against a reference signal comprising hemoglobin concentration, total protein concentration, or a combination thereof, to generate a within-individual normalized biomarker value. The normalization accounts for variations in menstrual fluid composition, flow rate, and sample volume between uses and between individuals, improving the accuracy and reproducibility of biomarker measurements for longitudinal tracking.

In some aspects, the lateral flow immunoassay includes a capillary-driven immunochromatographic assay.

In some aspects, the microfluidic system includes active gating via the hydrophilic nanoporous lipid-based membrane for volume control.

In some aspects, the method further includes transmitting data corresponding to the visible result to a mobile device application via a near-field communication (NFC) chip.

In some aspects, the detecting comprises processing the filtered menstrual fluid using a lateral flow immunoassay embedded in a nitrocellulose membrane.

In some aspects, the detecting comprises measuring an electrical signal via one or more electrochemical sensors correlated with the concentration of one or more target analytes.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 is a top view diagram of an example of a menstrual garment attachment with a channel entrance.

FIG. 2A is a diagram illustrating components of an example pad attachment that includes a microfluidic system and a blood reservoir system.

FIG. 2B is a diagram illustrating blood flow through the menstrual garment attachment of FIG. 2A.

FIG. 3 is a diagram illustrating an example of a transparent polymer covering a lateral flow immunoassay (“LFIA”). The transparent polymer covers and prevents contamination of the diagnostic apparatus while allowing the LFIA reading of the test results to be visible. These results are shown on the right garment attachment diagram after a period of time (e.g., 15-20 minutes) by displaying test/control lines.

FIG. 4 is a diagram illustrating an example of a microfluidic design configured for capillary flow.

FIG. 5 is a diagram illustrating an example of how the attachment adheres to the menstrual garment via an adhesive on the back of the attachment that causes it to stick to the garment.

### DETAILED DESCRIPTION

### Illustrative Numerical Examples

The following numerical values are provided as non-limiting illustrative examples only and are not intended to limit the scope of the claims. In some examples, the visible result may be available within about 10-30 minutes of menstrual fluid contact with the inlet, such as within about 15-20 minutes. In some examples, the hydrophilic nanoporous lipid-based membrane may rupture between about 60-120 μL of sample flow. In some examples, the filtering may exclude particles above about 5 μm in diameter. These numerical values represent illustrative embodiments and may be adjusted based on specific design requirements, biomarker targets, and manufacturing considerations. The actual values employed in any particular implementation may vary from these illustrative examples while still achieving the functional objectives described herein.

As used herein, the term “menstrual absorbent article” refers to any garment, article of clothing, or wearable insert configured to absorb, collect, or contain menstrual fluid in contact with the body. Menstrual absorbent articles include, but are not limited to, menstrual pads, sanitary napkins, panty liners, absorbent underwear, period underwear, absorbent activewear, absorbent swimwear, reusable cloth pads, and any other wearable article designed to manage menstrual fluid. The menstrual garment attachment of the present invention may be applied to any such menstrual absorbent article.

As used herein, the term “passively convey” refers to the transport of menstrual fluid through the device without requiring external power sources, pumps, or active mechanical components. Passive conveyance may be achieved through capillary action, wicking, absorption, gravity-assisted flow, or combinations thereof. The fluid transport system of the menstrual garment attachment is configured to passively convey menstrual fluid from a garment-facing surface of the housing to the detection module.

As used herein, the terms “diagnostic” and “wellness” are used interchangeably throughout this application. The menstrual garment attachment of the present invention can provide readings beneficial to both diagnostic purposes (such as detecting biomarkers associated with disease conditions) and wellness purposes (such as longitudinal biomarker tracking for personalized health monitoring). The device is not limited to either diagnostic or wellness applications and may be used for any purpose involving the detection or analysis of biomarkers in menstrual fluid.

The following description sets forth exemplary aspects of the present disclosure. It should be recognized, however, that such description is not intended as a limitation on the scope of the present disclosure. Rather, the description also encompasses combinations and modifications to those exemplary aspects described herein.

The term “menstrual garment attachment” is used herein to refer to a removable diagnostic device configured to be removably attached to a menstrual garment such as a sanitary pad. It is not integrated into or forms a structural component of the menstrual garment itself. Rather, the menstrual garment attachment is a standalone diagnostic accessory that can be applied to existing menstrual products through an adhesive backing or other attachment mechanism. The menstrual garment attachment may also be referred to as a “smart sticker” or “diagnostic patch.” Unlike collection-only attachments that transfer a menstrual fluid sample to an external laboratory for analysis, the menstrual garment attachment of the present disclosure performs at least one analytical or detection step within the device itself, producing a user-readable output at the point of use without requiring sample transfer or remote laboratory processing. The diagnostic components of the menstrual garment attachment are not integrated into or sandwiched between absorbent pad layers.

As used herein, the term “about” refers to plus or minus 10% of a given value.

In an example, a menstrual garment attachment for detecting disease biomarkers in menstrual fluid includes a translucent polymer layer positioned over a first portion of a lateral flow immunoassay and exposing a second portion of the lateral flow immunoassay. The menstrual garment attachment includes a surface layer configured to receive menstrual fluid. A distribution layer is positioned beneath the surface layer and configured to direct menstrual fluid toward the lateral flow immunoassay. An absorbent layer is positioned beneath the distribution layer. A waterproof layer comprising an antimicrobial material is included. The lateral flow immunoassay is positioned between the surface layer and the translucent polymer layer.

In an example, the menstrual garment attachment provides non-invasive detection of disease biomarkers using menstrual fluid as an analyte. Menstrual fluid is a discharged fluid that does not require invasive collection procedures. The menstrual garment attachment eliminates the need for vaginal swabbing or tissue sampling. Users are not required to perform uncomfortable or painful specimen collection procedures.

In an example, the menstrual garment attachment provides lab-free detection of disease biomarkers. The menstrual garment attachment produces visible results directly on the device without requiring samples to be shipped to laboratory facilities. The menstrual garment attachment does not require laboratory infrastructure for sample processing. The menstrual garment attachment does not require physical labor associated with laboratory testing. The menstrual garment attachment reduces costs associated with laboratory processing.

In an example, the menstrual garment attachment is configured as a removable top-sheet patch rather than having sensors embedded within absorbent layers. Sensors embedded within absorbent layers require integration into the pad structure itself. The menstrual garment attachment maintains structural separation from the menstrual pad. The menstrual garment attachment has its own backing. The menstrual garment attachment is not sandwiched between pad layers.

In an example, the menstrual garment attachment uses passive colorimetric detection rather than electrochemical detection. Electrochemical detection systems require laboratory analysis of collected samples. The menstrual garment attachment provides instant visual readouts on the device. The menstrual garment attachment does not require electrochemical sensors. The menstrual garment attachment does not require electronic components for detection.

In an example, the menstrual garment attachment provides instant visual readouts rather than remote laboratory analysis of dried blood spots. Remote laboratory analysis requires shipping samples to laboratory facilities. Remote laboratory analysis requires waiting for laboratory processing. The menstrual garment attachment provides results within minutes of menstrual fluid contact. The menstrual garment attachment does not require dried blood spot collection. The menstrual garment attachment does not require sample shipping.

In an example, the menstrual garment attachment is configured for single use and disposal. The menstrual garment attachment does not require sanitization between uses. Reusable diagnostic pad designs require users to properly sanitize pads to prevent infections. Proper sanitization is challenging for users who do not have access to sanitization resources. The menstrual garment attachment eliminates the need for sanitization. The menstrual garment attachment reduces infection risk associated with improper sanitization of reusable devices.

In an example, the menstrual garment attachment is configured for use in underserved communities. The menstrual garment attachment does not require access to laboratory facilities. The menstrual garment attachment does not require shipping infrastructure. The menstrual garment attachment does not require specialized medical equipment. The menstrual garment attachment provides accessible diagnostic testing for populations lacking laboratory infrastructure.

In an example, the menstrual garment attachment reduces barriers to cervical cancer screening. The menstrual garment attachment eliminates language barriers associated with complex testing procedures. The menstrual garment attachment reduces social stigma associated with invasive screenings. The menstrual garment attachment does not require specialized education for self-administration. The menstrual garment attachment provides a low-cost diagnostic tool for early detection of disease biomarkers.

### Menstrual Garment Attachment Structure And Layers

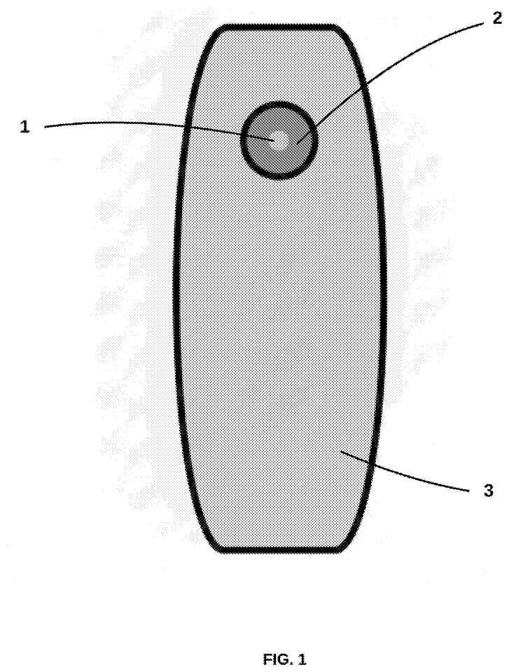

Referring to FIG. 1, a menstrual garment attachment for detecting disease biomarkers in menstrual fluid includes a translucent polymer layer positioned over a first portion of a lateral flow immunoassay and exposing a second portion of the lateral flow immunoassay. The translucent polymer layer covers and prevents contamination of the diagnostic apparatus while allowing the lateral flow immunoassay reading to be visible. The translucent polymer layer is positioned such that the first portion of the lateral flow immunoassay is protected from external contamination. The second portion of the lateral flow immunoassay is exposed to allow users to view test results.

In an example, the menstrual garment attachment includes a surface layer configured to receive menstrual fluid. The surface layer is composed of textile material. As shown in FIG. 1, cotton on the outside 3 is positioned on an exterior portion of the menstrual garment attachment. The cotton on the outside 3 enhances absorption of menstrual fluid. The surface layer quickly draws and retains menstrual fluid. The surface layer allows for quick absorption. The surface layer benefits the user by absorbing large quantities of fluid and helping maintain dryness on the surface. The surface layer is breathable and allows for constant air circulation while maintaining absorbency. The surface layer is hydrophilic to ensure menstrual blood flows through quickly and is completely absorbed. The surface layer comprises small pores for blood to pass through to microfluidic channels.

In some examples, the cotton on the outside 3 is replaced with bamboo fibers. Bamboo fibers have natural antibacterial properties. Bamboo fibers provide a sustainable alternative to cotton. Bamboo fibers quickly draw and retain fluid while providing antibacterial protection.

With continued reference to FIG. 1, a channel entrance 1 is positioned on the menstrual garment attachment. The channel entrance 1 provides an inlet for menstrual fluid to flow into the internal microfluidic system. A red circle 2 is positioned on the menstrual garment attachment to indicate the location of the channel entrance 1. The top of the menstrual garment attachment is coated with hydrophobic and hydrophilic coatings to ensure flow to the correct area. The hydrophilic coatings attract and direct menstrual fluid toward the channel entrance 1. The hydrophobic coatings act as barriers to prevent fluid from moving in undesired directions.

In an example, the menstrual garment attachment includes a distribution layer positioned beneath the surface layer. The distribution layer is configured to direct menstrual fluid toward the lateral flow immunoassay. The distribution layer moves menstrual fluid in a directed manner to detection and filtration areas. The distribution layer allows plasma and blood to easily flow through and make its way through the rest of the system.

In an example, the menstrual garment attachment includes an absorbent layer positioned beneath the distribution layer. The absorbent layer holds and distributes menstrual fluid. In some examples, the absorbent layer comprises an absorbent core made from superabsorbent polymer. The superabsorbent polymer prevents leakage. The absorbent core distributes menstrual fluid throughout the menstrual garment attachment.

In an example, the menstrual garment attachment includes a waterproof layer comprising an antimicrobial material. The waterproof layer prevents menstrual fluid from leaking through the menstrual garment attachment. The antimicrobial material provides antibacterial protection. In some examples, the antimicrobial material comprises charcoal bamboo fiber fabric. The charcoal bamboo fiber fabric provides natural antibacterial properties. The charcoal bamboo fiber fabric provides a sustainable material option for the waterproof layer.

In an example, the lateral flow immunoassay is positioned between the surface layer and the translucent polymer layer. The lateral flow immunoassay receives filtered menstrual fluid from the distribution layer. The lateral flow immunoassay detects biomarkers that react with certain components in the menstrual fluid. The lateral flow immunoassay allows for detection of infections or hormonal imbalances. The lateral flow immunoassay provides results based on the fluid's composition.

### Further Embodiments and Alternative Implementations

In an example, the detection module comprises a lateral flow immunoassay connected to a near-field communication (NFC) chip for wireless digital readout. The NFC chip is configured to transmit analytical data from the lateral flow immunoassay to a mobile device application. An optical reader may be coupled to the NFC chip and configured to interpret the colorimetric results of the lateral flow immunoassay. The optical reader converts the colorimetric results to digital data for transmission via the NFC chip.

In an example, the detection module comprises one or more electrochemical sensors connected to an NFC chip or Bluetooth low energy (BLE) transmitter. The electrochemical sensors may include amperometric sensors, impedance sensors, or field-effect transistor-based sensors configured to generate an electrical signal correlated with the concentration of one or more target analytes in menstrual fluid. The electrical signals are processed and transmitted via the NFC chip or BLE transmitter to the mobile device application for display and storage.

In an example, the menstrual garment attachment includes a microprocessor or application-specific integrated circuit (ASIC) for on-device signal processing before wireless transmission. The microprocessor or ASIC processes raw sensor data to generate analytical results prior to transmission to the mobile device application. The on-device signal processing reduces the computational burden on the mobile device and enables faster result display.

In an example, the menstrual garment attachment includes a small battery or energy harvesting element to power the electronic detection module. The battery may comprise a thin-film battery or printed battery configured to fit within the housing of the menstrual garment attachment. The energy harvesting element may comprise a piezoelectric element or thermoelectric element configured to generate electrical power from user movement or body heat.

In an example, multiple menstrual garment attachments are used sequentially across menstrual cycles with a companion mobile device application aggregating and trending results. The mobile device application stores data from each menstrual garment attachment used by the user. The mobile device application displays longitudinal trends in biomarker levels across multiple menstrual cycles. The longitudinal tracking enables personalized trend analysis by comparing current results to previous results from the same user.

In an example, the menstrual garment attachment includes a QR code or NFC tag that links to a user-specific health record or telehealth consultation interface. The QR code or NFC tag is scannable by a mobile device. The QR code or NFC tag directs the user to a secure health record portal or telehealth consultation platform. The linkage to the health record or telehealth interface enables users to share diagnostic results with healthcare providers.

In an example, the menstrual garment attachment includes wings extending from opposite sides of the menstrual garment attachment. The wings extend laterally from a first side of the menstrual garment attachment and from a second side of the menstrual garment attachment. The wings are configured to wrap around underwear worn by a user. The wings provide additional securement of the menstrual garment attachment to the user's underwear. The wings prevent the menstrual garment attachment from shifting during user movement. The wings extend outward from a central body portion of the menstrual garment attachment. The wings are positioned at a location along the menstrual garment attachment that corresponds to the sides of a user's underwear.

In an example, the wings are configured to fold underneath the user's underwear. The wings wrap from the top surface of the menstrual garment attachment, around the edges of the underwear, and underneath the underwear. The wings contact the exterior surface of the underwear when folded underneath. The folding of the wings around the underwear creates a secure attachment between the menstrual garment attachment and the underwear. The wings maintain the position of the menstrual garment attachment relative to the underwear during user activity.

In some examples, the menstrual garment attachment includes clasps configured to secure the wings. The clasps are positioned on the wings. The clasps engage with corresponding portions of the wings or the menstrual garment attachment to hold the wings in a secured position. The clasps prevent the wings from unfolding during use. The clasps maintain the wrapped configuration of the wings around the underwear.

In some examples, the clasps comprise snap fasteners. The snap fasteners include a first snap component positioned on a first surface of the wing and a second snap component positioned on a second surface configured to receive the first snap component. The first snap component and the second snap component engage when pressed together. The snap fasteners provide a secure connection that resists separation during user movement.

In some examples, the clasps comprise hook and loop fasteners. The hook and loop fasteners include a hook component positioned on a first surface of the wing and a loop component positioned on a second surface configured to receive the hook component. The hook component and the loop component engage when pressed together. The hook and loop fasteners provide an adjustable connection that allows users to customize the tightness of the wing securement.

In some examples, the clasps comprise adhesive tabs. The adhesive tabs are positioned on the wings. The adhesive tabs include an adhesive surface configured to adhere to the exterior surface of the underwear or to a corresponding surface on the menstrual garment attachment. The adhesive tabs hold the wings in the wrapped configuration around the underwear.

In an example, the wings and the clasps secure the menstrual garment attachment during use. The wings provide lateral securement to the user's underwear. The combination of the wings and the clasps provides multiple points of attachment that maintain the position of the menstrual garment attachment during user activity.

In an example, the wings extend from the menstrual garment attachment at a position that aligns with the leg openings of the user's underwear. The wings wrap around the underwear at the leg openings. The positioning of the wings at the leg openings provides securement at a location where the underwear contacts the user's thighs. The securement at the leg openings prevents the menstrual garment attachment from bunching or shifting toward the front or back of the underwear.

In some examples, the wings comprise the same material as the waterproof layer of the menstrual garment attachment. The wings provide a waterproof barrier that extends beyond the central body portion of the menstrual garment attachment. The waterproof material of the wings prevents menstrual fluid from leaking around the edges of the menstrual garment attachment.

In some examples, the wings comprise the antimicrobial material. The antimicrobial material of the wings provides antibacterial protection at the wing portions of the menstrual garment attachment. The antimicrobial material of the wings reduces bacterial growth at the portions of the menstrual garment attachment that contact the exterior surface of the underwear.

### Translucent Polymer Layer and Sealing

In an example, the menstrual garment attachment includes a translucent polymer layer positioned over a first portion of a lateral flow immunoassay. The translucent polymer layer exposes a second portion of the lateral flow immunoassay. The translucent polymer layer covers and prevents contamination of the diagnostic apparatus. The translucent polymer layer allows the lateral flow immunoassay reading to be visible to a user. The translucent polymer layer protects the lateral flow immunoassay from external contaminants including dust, debris, and moisture. The translucent polymer layer maintains the integrity of the lateral flow immunoassay during use and handling. The translucent polymer layer provides a barrier between the external environment and the diagnostic components of the menstrual garment attachment.

In an example, the translucent polymer layer is positioned such that the first portion of the lateral flow immunoassay remains protected while the second portion of the lateral flow immunoassay remains visible for result interpretation. The first portion of the lateral flow immunoassay includes components that are susceptible to contamination. The second portion of the lateral flow immunoassay includes the results region where test lines and control lines appear. The positioning of the translucent polymer layer over the first portion and exposing the second portion allows users to view test results without compromising the diagnostic accuracy of the lateral flow immunoassay.

In some examples, the translucent polymer layer is bonded to the surface layer and to the lateral flow immunoassay to form a seal. The bonding of the translucent polymer layer to the surface layer creates a sealed interface between the translucent polymer layer and the surface layer. The bonding of the translucent polymer layer to the lateral flow immunoassay creates a sealed interface between the translucent polymer layer and the lateral flow immunoassay. The seal formed by the bonding prevents menstrual fluid from bypassing the intended fluid pathway. The seal formed by the bonding prevents external contaminants from entering the diagnostic zone. The seal formed by the bonding maintains the sterility of the lateral flow immunoassay components. The seal formed by the bonding prevents leakage of menstrual fluid from the menstrual garment attachment. The bonding of the translucent polymer layer to the surface layer and to the lateral flow immunoassay provides structural integrity to the menstrual garment attachment.

In some examples, the bonding of the translucent polymer layer to the surface layer and to the lateral flow immunoassay comprises thermal bonding. Thermal bonding applies heat to fuse the translucent polymer layer to the surface layer and to the lateral flow immunoassay. In some examples, the bonding of the translucent polymer layer to the surface layer and to the lateral flow immunoassay comprises adhesive bonding. Adhesive bonding applies an adhesive material between the translucent polymer layer and the surface layer and between the translucent polymer layer and the lateral flow immunoassay. In some examples, the bonding of the translucent polymer layer to the surface layer and to the lateral flow immunoassay comprises ultrasonic bonding. Ultrasonic bonding applies ultrasonic vibrations to fuse the translucent polymer layer to the surface layer and to the lateral flow immunoassay.

In some examples, the translucent polymer layer comprises a hydrophobic polymer. The hydrophobic polymer repels water and aqueous fluids. The hydrophobic polymer prevents menstrual fluid from adhering to the exterior surface of the translucent polymer layer. The hydrophobic polymer maintains visibility of the lateral flow immunoassay results by preventing fluid accumulation on the viewing surface. The hydrophobic polymer prevents moisture from penetrating through the translucent polymer layer to the lateral flow immunoassay. The hydrophobic polymer provides a barrier that resists fluid penetration while maintaining optical clarity for result visualization.

In some examples, the hydrophobic polymer comprises polyisoprene. Polyisoprene is a synthetic rubber material with hydrophobic properties. Polyisoprene provides flexibility to the translucent polymer layer. Polyisoprene provides durability to the translucent polymer layer. Polyisoprene maintains optical clarity for visualization of the lateral flow immunoassay results. Polyisoprene is biocompatible for contact with skin and menstrual fluid. Polyisoprene provides resistance to degradation from exposure to menstrual fluid. Polyisoprene maintains the seal formed by the bonding of the translucent polymer layer to the surface layer and to the lateral flow immunoassay during use.

In some examples, the hydrophobic polymer comprises materials other than polyisoprene. In some examples, the hydrophobic polymer comprises silicone. In some examples, the hydrophobic polymer comprises polyethylene. In some examples, the hydrophobic polymer comprises polypropylene. The selection of the hydrophobic polymer material is based on the desired optical clarity, flexibility, durability, and biocompatibility for the menstrual garment attachment application.

In an example, the translucent polymer layer allows colorimetric nanoparticles of the lateral flow immunoassay to be visible through the translucent polymer layer. The colorimetric nanoparticles produce color changes at test lines and control lines of the lateral flow immunoassay. The translucent polymer layer transmits light to allow users to observe the color changes. The translucent polymer layer does not distort or obscure the color changes produced by the colorimetric nanoparticles. The translucent polymer layer provides a transparent window for viewing the lateral flow immunoassay results.

In an example, the translucent polymer layer is configured to withstand the conditions of use of the menstrual garment attachment. The translucent polymer layer maintains the seal during user movement. The translucent polymer layer maintains the seal during exposure to body heat. The translucent polymer layer maintains the seal during exposure to moisture. The translucent polymer layer maintains optical clarity throughout the duration of use of the menstrual garment attachment.

### Lateral Flow Immunoassay Components

In an example, the menstrual garment attachment includes a lateral flow immunoassay positioned between the surface layer and the translucent polymer layer. The lateral flow immunoassay receives filtered menstrual fluid from the distribution layer. The lateral flow immunoassay detects biomarkers that react with certain components in the menstrual fluid. The lateral flow immunoassay allows for detection of infections or hormonal imbalances. The lateral flow immunoassay provides results based on the fluid's composition. The lateral flow immunoassay utilizes capillary forces within the microfluidic system to ensure that the correct volume of fluid reaches the test area.

In some examples, the lateral flow immunoassay is disposed within a plastic casing. The plastic casing provides structural support for the lateral flow immunoassay. The plastic casing protects the lateral flow immunoassay from physical damage during handling and use. The plastic casing maintains the alignment of the components of the lateral flow immunoassay. The plastic casing prevents contamination of the lateral flow immunoassay components from external sources. The plastic casing provides a defined housing that positions the lateral flow immunoassay between the surface layer and the translucent polymer layer.

In an example, the lateral flow immunoassay comprises a collection pad positioned at a first end of the lateral flow immunoassay. The collection pad receives menstrual fluid that enters the lateral flow immunoassay. The collection pad is positioned at the first end of the lateral flow immunoassay to serve as the initial point of contact for menstrual fluid. The collection pad absorbs menstrual fluid from the distribution layer. The collection pad holds menstrual fluid for transfer to subsequent components of the lateral flow immunoassay. The collection pad is configured to receive a volume of menstrual fluid sufficient for biomarker detection.

In some examples, the lateral flow immunoassay further comprises a sample pad. The sample pad receives menstrual fluid from the collection pad. The sample pad is positioned downstream from the collection pad in the fluid flow path. The sample pad prepares the menstrual fluid sample for interaction with detection reagents. The sample pad filters or conditions the menstrual fluid prior to biomarker detection.

In some examples, the lateral flow immunoassay further comprises a conjugate pad. The conjugate pad is positioned downstream from the sample pad in the fluid flow path. The conjugate pad contains detection reagents. The conjugate pad contains labeled antibodies or other binding molecules configured to bind to target biomarkers. The conjugate pad releases the labeled antibodies or other binding molecules when menstrual fluid contacts the conjugate pad. The labeled antibodies or other binding molecules bind to target biomarkers present in the menstrual fluid. The conjugate pad initiates the detection reaction for biomarker identification.

In some examples, the lateral flow immunoassay further comprises a results region comprising test lines and control lines. The results region is positioned downstream from the conjugate pad in the fluid flow path. The test lines contain immobilized capture molecules configured to bind to biomarker-antibody complexes. The test lines produce visible color changes when biomarker-antibody complexes bind to the immobilized capture molecules. The control lines contain immobilized molecules configured to bind to labeled antibodies regardless of biomarker presence. The control lines produce visible color changes to indicate that the lateral flow immunoassay has functioned properly. The results region provides visual indication of biomarker presence or absence based on the color changes at the test lines and the control lines.

In some examples, the lateral flow immunoassay further comprises a wick pad. The wick pad is positioned at a second end of the lateral flow immunoassay opposite the first end where the collection pad is positioned. The wick pad absorbs excess menstrual fluid that has passed through the results region. The wick pad maintains fluid flow through the lateral flow immunoassay by drawing menstrual fluid through the system via capillary action. The wick pad prevents backflow of menstrual fluid toward the results region. The wick pad provides a reservoir for menstrual fluid that has completed the detection process.

In some examples, the lateral flow immunoassay is configured as a membrane-based binding assay. The membrane-based binding assay utilizes a porous membrane substrate for biomarker detection. The membrane-based binding assay relies on binding interactions between biomarkers and immobilized capture molecules on the membrane substrate. The membrane-based binding assay provides an alternative terminology for describing the lateral flow immunoassay detection mechanism. The membrane-based binding assay operates through capillary-driven flow of menstrual fluid across the membrane substrate.

In some examples, the lateral flow immunoassay includes a nitrocellulose membrane. The nitrocellulose membrane provides the substrate for the results region. The nitrocellulose membrane has a porous structure that naturally moves fluid. The nitrocellulose membrane moves menstrual blood samples toward the detection zone through capillary action. The nitrocellulose membrane supports the immobilized capture molecules at the test lines and the control lines.

In some examples, colored nanoparticles are embedded onto the nitrocellulose membrane. The colored nanoparticles create color changes when biomarkers bind. The colored nanoparticles are conjugated to antibodies or other binding molecules. The colored nanoparticles accumulate at the test lines when biomarker-antibody complexes bind to the immobilized capture molecules. The colored nanoparticles produce visible color changes at the test lines that indicate biomarker presence. The colored nanoparticles accumulate at the control lines when labeled antibodies bind to the immobilized molecules. The colored nanoparticles produce visible color changes at the control lines that indicate proper function of the lateral flow immunoassay. The colored nanoparticles are visible through the translucent polymer layer as described previously.

In some examples, the colored nanoparticles comprise gold nanoparticles. Gold nanoparticles produce a red or pink color at the test lines and the control lines. In some examples, the colored nanoparticles comprise latex nanoparticles. Latex nanoparticles are available in various colors for multi-zone detection applications. In some examples, the colored nanoparticles comprise carbon nanoparticles. Carbon nanoparticles produce a black or dark gray color at the test lines and the control lines.

In an example, when menstrual blood reaches the results region, specific biomarkers such as reproductive hormones, proteins, or pathogens bind to the antibodies conjugated to the colored nanoparticles. The binding of the biomarkers to the antibodies creates biomarker-antibody complexes. The biomarker-antibody complexes migrate to the test lines. The biomarker-antibody complexes bind to the immobilized capture molecules at the test lines. The accumulation of the colored nanoparticles at the test lines creates color changes visible as test results. The color changes at the test lines indicate the presence of the specific biomarkers in the menstrual fluid sample.

### Electronic Sensor and NFC Embodiments

In an example, the menstrual garment attachment includes an electronic sensor as an alternative to or in addition to the lateral flow immunoassay for biomarker detection. The electronic sensor is disposed within the housing of the menstrual garment attachment. The electronic sensor is configured to detect one or more disease biomarkers in menstrual fluid. The electronic sensor may comprise an electrochemical sensor, an optical sensor, or other sensor types suitable for biomarker detection in biological fluids.

In an example, the menstrual garment attachment includes a near-field communication (NFC) chip disposed within the housing. The NFC chip is operatively coupled to the electronic sensor or to a reader configured to interpret results from the lateral flow immunoassay. The NFC chip is configured to transmit data corresponding to the detected one or more disease biomarkers to a mobile device application. The NFC chip enables wireless data transmission without requiring batteries or active power sources within the menstrual garment attachment.

In an example, the mobile device application receives data from the NFC chip when a user's mobile device is placed in proximity to the menstrual garment attachment. The mobile device application is configured to display the detected biomarker results to the user. The mobile device application is configured to store data corresponding to the detected one or more disease biomarkers for longitudinal tracking. The longitudinal tracking enables personalized trend analysis by comparing current results to previous results from the same user over multiple menstrual cycles.

In an example, the electronic sensor embodiment provides an alternative detection mechanism to the lateral flow immunoassay. The electronic sensor may provide quantitative biomarker measurements rather than qualitative colorimetric results. The electronic sensor may detect biomarkers through electrochemical reactions that produce measurable electrical signals. The electrical signals are processed and transmitted via the NFC chip to the mobile device application for display and storage.

In an example, the menstrual garment attachment combines the lateral flow immunoassay with the NFC chip for data transmission. The NFC chip may be coupled to an optical reader configured to interpret the colorimetric results of the lateral flow immunoassay. The optical reader converts the colorimetric results to digital data for transmission via the NFC chip. The digital data is transmitted to the mobile device application for display, storage, and longitudinal tracking.

In an example, the menstrual pad provides an integrated diagnostic solution for detecting disease biomarkers in menstrual fluid. The menstrual pad combines the absorbent functionality of a conventional menstrual pad with the diagnostic capabilities of the lateral flow immunoassay. The menstrual pad eliminates the need for a separate diagnostic attachment. The menstrual pad provides a single-use diagnostic product that is disposed of after use.

In an example, the translucent polymer layer of the menstrual pad is positioned over the first portion of the lateral flow immunoassay. The translucent polymer layer covers and protects the first portion of the lateral flow immunoassay from contamination. The translucent polymer layer exposes the second portion of the lateral flow immunoassay. The second portion of the lateral flow immunoassay includes the results region where test lines and control lines appear. The translucent polymer layer allows users to view test results through the translucent polymer layer. The translucent polymer layer maintains optical clarity for visualization of the lateral flow immunoassay results.

In an example, the surface layer of the menstrual pad is configured to receive menstrual fluid. The surface layer is positioned at the top of the menstrual pad in contact with the user's body. The surface layer absorbs menstrual fluid upon contact. The surface layer is composed of textile material that enhances absorption. The surface layer is hydrophilic to ensure menstrual blood flows through quickly and is completely absorbed. The surface layer comprises small pores for blood to pass through to the distribution layer.

In an example, the distribution layer of the menstrual pad is positioned beneath the surface layer. The distribution layer is configured to direct menstrual fluid toward the lateral flow immunoassay. The distribution layer moves menstrual fluid in a directed manner from the surface layer to the lateral flow immunoassay. The distribution layer distributes menstrual fluid across the menstrual pad to ensure adequate fluid reaches the lateral flow immunoassay for biomarker detection.

In an example, the absorbent layer of the menstrual pad is positioned beneath the distribution layer. The absorbent layer holds and distributes menstrual fluid. The absorbent layer absorbs excess menstrual fluid that is not directed to the lateral flow immunoassay. The absorbent layer prevents leakage of menstrual fluid from the menstrual pad. The absorbent layer maintains dryness on the surface of the menstrual pad.

In an example, the waterproof layer of the menstrual pad comprises an antimicrobial material. The waterproof layer is positioned beneath the absorbent layer. The waterproof layer prevents menstrual fluid from leaking through the bottom of the menstrual pad. The antimicrobial material provides antibacterial protection. The antimicrobial material reduces bacterial growth within the menstrual pad during use.

In an example, the lateral flow immunoassay of the menstrual pad is positioned between the surface layer and the translucent polymer layer. The lateral flow immunoassay receives filtered menstrual fluid from the distribution layer. The lateral flow immunoassay detects biomarkers that react with certain components in the menstrual fluid. The lateral flow immunoassay provides results based on the fluid's composition. The lateral flow immunoassay produces visible color changes at test lines and control lines to indicate biomarker presence or absence.

In an example, the pair of wings extends from opposite sides of the menstrual pad. A first wing extends from a first side of the menstrual pad. A second wing extends from a second side of the menstrual pad opposite the first side. The pair of wings is configured to wrap around underwear worn by a user. The pair of wings provides securement of the menstrual pad to the user's underwear. The pair of wings prevents the menstrual pad from shifting during user movement. The pair of wings extends outward from a central body portion of the menstrual pad.

In an example, the pair of wings is configured to fold underneath the user's underwear. The pair of wings wraps from the top surface of the menstrual pad, around the edges of the underwear, and underneath the underwear. The pair of wings contacts the exterior surface of the underwear when folded underneath. The folding of the pair of wings around the underwear creates a secure attachment between the menstrual pad and the underwear.

In some examples, clasps are configured to secure the pair of wings. The clasps are positioned on the pair of wings. The clasps engage with corresponding portions of the pair of wings or the menstrual pad to hold the pair of wings in a secured position. The clasps prevent the pair of wings from unfolding during use. The clasps maintain the wrapped configuration of the pair of wings around the underwear.

In some examples, the clasps comprise snap fasteners. The snap fasteners include a first snap component positioned on a first surface of each wing and a second snap component positioned on a second surface configured to receive the first snap component. The first snap component and the second snap component engage when pressed together. The snap fasteners provide a secure connection that resists separation during user movement.

In some examples, the clasps comprise hook and loop fasteners. The hook and loop fasteners include a hook component positioned on a first surface of each wing and a loop component positioned on a second surface configured to receive the hook component. The hook component and the loop component engage when pressed together. The hook and loop fasteners provide an adjustable connection that allows users to customize the tightness of the wing securement.

In some examples, the clasps comprise adhesive tabs. The adhesive tabs are positioned on the pair of wings. The adhesive tabs include an adhesive surface configured to adhere to the exterior surface of the underwear or to a corresponding surface on the menstrual pad. The adhesive tabs hold the pair of wings in the wrapped configuration around the underwear.

In some examples, the translucent polymer layer comprises a hydrophobic polymer. The hydrophobic polymer repels water and aqueous fluids. The hydrophobic polymer prevents menstrual fluid from adhering to the exterior surface of the translucent polymer layer. The hydrophobic polymer maintains visibility of the lateral flow immunoassay results by preventing fluid accumulation on the viewing surface. The hydrophobic polymer prevents moisture from penetrating through the translucent polymer layer to the lateral flow immunoassay.

In some examples, the hydrophobic polymer comprises polyisoprene. Polyisoprene is a synthetic rubber material with hydrophobic properties. Polyisoprene provides flexibility to the translucent polymer layer. Polyisoprene provides durability to the translucent polymer layer. Polyisoprene maintains optical clarity for visualization of the lateral flow immunoassay results. Polyisoprene is biocompatible for contact with skin and menstrual fluid.

In some examples, the lateral flow immunoassay comprises a collection pad positioned at a first end of the lateral flow immunoassay. The collection pad receives menstrual fluid that enters the lateral flow immunoassay. The collection pad is positioned at the first end of the lateral flow immunoassay to serve as the initial point of contact for menstrual fluid. The collection pad absorbs menstrual fluid from the distribution layer. The collection pad holds menstrual fluid for transfer to subsequent components of the lateral flow immunoassay. The collection pad is configured to receive a volume of menstrual fluid sufficient for biomarker detection.

### Menstrual Pad With Antimicrobial Waterproof Layer Covering

In an example, a menstrual pad for detecting disease biomarkers in menstrual fluid includes a translucent polymer layer positioned over a first portion of a lateral flow immunoassay and exposing a second portion of the lateral flow immunoassay. The menstrual pad includes a surface layer configured to receive menstrual fluid. A distribution layer is positioned beneath the surface layer and configured to direct menstrual fluid toward the lateral flow immunoassay. An absorbent layer is positioned beneath the distribution layer. A waterproof layer comprising an antimicrobial material is included in the menstrual pad. The lateral flow immunoassay is positioned between the surface layer and the translucent polymer layer. A pair of wings extends from opposite sides of the menstrual pad. The waterproof layer comprising the antimicrobial material covers the menstrual pad and the pair of wings.

In an example, the waterproof layer comprising the antimicrobial material extends across the entire bottom surface of the menstrual pad. The waterproof layer comprising the antimicrobial material extends from the central body portion of the menstrual pad to the pair of wings. The waterproof layer comprising the antimicrobial material provides continuous coverage across the menstrual pad and the pair of wings. The continuous coverage of the waterproof layer comprising the antimicrobial material prevents menstrual fluid from leaking through any portion of the menstrual pad or the pair of wings.

In an example, the waterproof layer comprising the antimicrobial material provides a barrier that prevents menstrual fluid from passing through the bottom of the menstrual pad. The waterproof layer comprising the antimicrobial material provides a barrier that prevents menstrual fluid from passing through the pair of wings. The waterproof layer comprising the antimicrobial material maintains the integrity of the menstrual pad during use. The waterproof layer comprising the antimicrobial material maintains the integrity of the pair of wings during use.

In an example, the antimicrobial material of the waterproof layer provides antibacterial protection across the menstrual pad and the pair of wings. The antimicrobial material reduces bacterial growth within the menstrual pad during use. The antimicrobial material reduces bacterial growth within the pair of wings during use. The antimicrobial material provides continuous antibacterial protection across the entire surface area covered by the waterproof layer.

In an example, the pair of wings extends from opposite sides of the menstrual pad. A first wing extends from a first side of the menstrual pad. A second wing extends from a second side of the menstrual pad opposite the first side. The waterproof layer comprising the antimicrobial material covers the first wing. The waterproof layer comprising the antimicrobial material covers the second wing. The coverage of the waterproof layer comprising the antimicrobial material on the pair of wings provides waterproof and antibacterial protection at the wing portions of the menstrual pad.

In an example, the pair of wings is configured to wrap around underwear worn by a user. The pair of wings contacts the exterior surface of the underwear when folded underneath. The waterproof layer comprising the antimicrobial material is positioned on the pair of wings such that the waterproof layer contacts the exterior surface of the underwear. The waterproof layer comprising the antimicrobial material prevents menstrual fluid from transferring to the underwear through the pair of wings. The antimicrobial material of the waterproof layer reduces bacterial growth at the portions of the pair of wings that contact the exterior surface of the underwear.

In some examples, the antimicrobial material comprises charcoal bamboo fiber fabric. The charcoal bamboo fiber fabric provides natural antibacterial properties. The charcoal bamboo fiber fabric provides a sustainable material option for the waterproof layer. The charcoal bamboo fiber fabric covers the menstrual pad and the pair of wings. The charcoal bamboo fiber fabric provides continuous antibacterial protection across the menstrual pad and the pair of wings. The charcoal bamboo fiber fabric reduces bacterial growth throughout the menstrual pad during use.

In some examples, the charcoal bamboo fiber fabric is combined with a waterproof backing material. The waterproof backing material provides the waterproof barrier function of the waterproof layer. The charcoal bamboo fiber fabric provides the antimicrobial function of the waterproof layer. The combination of the charcoal bamboo fiber fabric and the waterproof backing material provides both waterproof and antimicrobial protection across the menstrual pad and the pair of wings.

In some examples, the lateral flow immunoassay comprises a collection pad positioned at a first end of the lateral flow immunoassay. As described previously, the collection pad receives menstrual fluid that enters the lateral flow immunoassay. The collection pad is positioned at the first end of the lateral flow immunoassay to serve as the initial point of contact for menstrual fluid. The collection pad absorbs menstrual fluid from the distribution layer. The collection pad holds menstrual fluid for transfer to subsequent components of the lateral flow immunoassay. The collection pad is configured to receive a volume of menstrual fluid sufficient for biomarker detection.

In an example, the translucent polymer layer is positioned over the first portion of the lateral flow immunoassay. As described previously, the translucent polymer layer covers and protects the first portion of the lateral flow immunoassay from contamination. The translucent polymer layer exposes the second portion of the lateral flow immunoassay. The second portion of the lateral flow immunoassay includes the results region where test lines and control lines appear. The translucent polymer layer allows users to view test results through the translucent polymer layer.

In an example, the surface layer is configured to receive menstrual fluid. As described previously, the surface layer is positioned at the top of the menstrual pad in contact with the user's body. The surface layer absorbs menstrual fluid upon contact. The surface layer is composed of textile material that enhances absorption. The surface layer is hydrophilic to ensure menstrual blood flows through quickly and is completely absorbed.

In an example, the distribution layer is positioned beneath the surface layer. As described previously, the distribution layer is configured to direct menstrual fluid toward the lateral flow immunoassay. The distribution layer moves menstrual fluid in a directed manner from the surface layer to the lateral flow immunoassay. The distribution layer distributes menstrual fluid across the menstrual pad to ensure adequate fluid reaches the lateral flow immunoassay for biomarker detection.

In an example, the absorbent layer is positioned beneath the distribution layer. As described previously, the absorbent layer holds and distributes menstrual fluid. The absorbent layer absorbs excess menstrual fluid that is not directed to the lateral flow immunoassay. The absorbent layer prevents leakage of menstrual fluid from the menstrual pad. The absorbent layer maintains dryness on the surface of the menstrual pad.

In an example, the lateral flow immunoassay is positioned between the surface layer and the translucent polymer layer. As described previously, the lateral flow immunoassay receives filtered menstrual fluid from the distribution layer. The lateral flow immunoassay detects biomarkers that react with certain components in the menstrual fluid. The lateral flow immunoassay provides results based on the fluid's composition. The lateral flow immunoassay produces visible color changes at test lines and control lines to indicate biomarker presence or absence.

### User Instructions and Accessibility Features

In an example, a menstrual garment attachment includes a surface-mountable pictographic instruction label for user interpretation without literacy requirements. The surface-mountable pictographic instruction label is positioned on an exterior surface of the menstrual garment attachment. The surface-mountable pictographic instruction label comprises a series of pictographic images that illustrate the steps for using the menstrual garment attachment. The pictographic images depict the actions a user performs to apply, use, and interpret results from the menstrual garment attachment. The pictographic images do not include text. The pictographic images communicate instructions through visual representations of actions and objects. The surface-mountable pictographic instruction label allows users to understand how to use the menstrual garment attachment without reading written instructions.

In an example, the surface-mountable pictographic instruction label addresses language barriers in underserved communities. Users who speak different languages are able to interpret the pictographic images without translation. Users who are not literate are able to interpret the pictographic images without reading text. The surface-mountable pictographic instruction label eliminates the need for written instructions in multiple languages. The surface-mountable pictographic instruction label provides a universal communication method for users across different linguistic backgrounds.

In an example, the surface-mountable pictographic instruction label includes pictographic images depicting the application of the menstrual garment attachment to a menstrual pad or underwear. The pictographic images show the orientation of the menstrual garment attachment relative to the menstrual pad or underwear. The pictographic images show the positioning of the menstrual garment attachment on the menstrual pad or underwear. The pictographic images show the removal of any protective backing from an adhesive surface of the menstrual garment attachment. The pictographic images show the pressing of the menstrual garment attachment onto the menstrual pad or underwear to secure the menstrual garment attachment in position.

In an example, the surface-mountable pictographic instruction label includes pictographic images depicting the interpretation of test results from the lateral flow immunoassay. The pictographic images show the location of the results region on the menstrual garment attachment. The pictographic images show examples of positive test results with color changes at test lines. The pictographic images show examples of negative test results without color changes at test lines. The pictographic images show examples of control line color changes that indicate proper function of the lateral flow immunoassay. The pictographic images allow users to compare the appearance of the results region on the menstrual garment attachment to the examples depicted in the pictographic images.

In an example, the surface-mountable pictographic instruction label includes pictographic images depicting the timing for result interpretation. The pictographic images show a representation of time passing between application of the menstrual garment attachment and result interpretation. The pictographic images indicate the waiting period before results are available. The pictographic images communicate the timing information without numerical text through visual representations such as clock symbols or sequential images.

In an example, the surface-mountable pictographic instruction label is printed directly on the exterior surface of the menstrual garment attachment. In some examples, the surface-mountable pictographic instruction label is printed on a separate label that is adhered to the exterior surface of the menstrual garment attachment. In some examples, the surface-mountable pictographic instruction label is printed on packaging of the menstrual garment attachment. The surface-mountable pictographic instruction label is positioned in a location that is visible to the user during use of the menstrual garment attachment.

In some examples, the menstrual garment attachment includes a QR-linked visual guide for user interpretation. The QR-linked visual guide comprises a QR code printed on the menstrual garment attachment or on packaging of the menstrual garment attachment. The QR code is scannable by a mobile device with a camera. The QR code links to a visual guide accessible through the mobile device. The visual guide comprises video content, animated content, or image-based content that illustrates the steps for using the menstrual garment attachment. The visual guide does not require literacy for interpretation. The visual guide communicates instructions through visual demonstrations of actions and objects.

In some examples, the QR-linked visual guide addresses language barriers in underserved communities. The visual guide comprises video content or animated content that demonstrates the use of the menstrual garment attachment without spoken narration. The visual guide comprises video content or animated content that demonstrates the use of the menstrual garment attachment with visual cues rather than text overlays. Users who speak different languages are able to interpret the visual guide without translation. Users who are not literate are able to interpret the visual guide without reading text.

In some examples, the QR-linked visual guide is accessible without electronic tools beyond a mobile device with a camera. The QR code links to content that is viewable on the mobile device without additional software installation. The QR code links to content that is viewable on the mobile device without account creation or login. The QR code links to content that is viewable on the mobile device without payment or subscription. The QR-linked visual guide provides accessible instructions for users who have access to a mobile device with a camera.

In some examples, the QR-linked visual guide includes video content depicting the application of the menstrual garment attachment to a menstrual pad or underwear. The video content shows the orientation of the menstrual garment attachment relative to the menstrual pad or underwear. The video content shows the positioning of the menstrual garment attachment on the menstrual pad or underwear. The video content shows the removal of any protective backing from an adhesive surface of the menstrual garment attachment. The video content shows the pressing of the menstrual garment attachment onto the menstrual pad or underwear to secure the menstrual garment attachment in position.

In some examples, the QR-linked visual guide includes video content depicting the interpretation of test results from the lateral flow immunoassay. The video content shows the location of the results region on the menstrual garment attachment. The video content shows examples of positive test results with color changes at test lines. The video content shows examples of negative test results without color changes at test lines. The video content shows examples of control line color changes that indicate proper function of the lateral flow immunoassay. The video content allows users to compare the appearance of the results region on the menstrual garment attachment to the examples depicted in the video content.

In some examples, the QR-linked visual guide includes video content depicting the timing for result interpretation. The video content shows a representation of time passing between application of the menstrual garment attachment and result interpretation. The video content indicates the waiting period before results are available. The video content communicates the timing information through visual representations such as clock animations or time-lapse sequences.

In an example, the surface-mountable pictographic instruction label and the QR-linked visual guide are both included on the menstrual garment attachment or packaging of the menstrual garment attachment. The surface-mountable pictographic instruction label provides instructions for users who do not have access to a mobile device with a camera. The QR-linked visual guide provides more detailed instructions for users who have access to a mobile device with a camera. The combination of the surface-mountable pictographic instruction label and the QR-linked visual guide provides multiple options for user instruction that address different levels of technology access in underserved communities.

In an example, the surface-mountable pictographic instruction label and the QR-linked visual guide reduce barriers to cervical cancer screening in underserved communities. The surface-mountable pictographic instruction label and the QR-linked visual guide eliminate language barriers associated with complex testing procedures. The surface-mountable pictographic instruction label and the QR-linked visual guide reduce the need for specialized education for self-administration of the menstrual garment attachment. The surface-mountable pictographic instruction label and the QR-linked visual guide provide accessible diagnostic testing instructions for populations with varying literacy levels and language backgrounds.

### Method For Detecting Reproductive Health Biomarkers—Microfluidic Channeling

In an example, a method for detecting one or more reproductive health biomarkers using menstrual fluid collected within a menstrual garment attachment includes channeling menstrual fluid from a pad-facing inlet to an internal microfluidic system. The internal microfluidic system comprises capillary-actuated polydimethylsiloxane (PDMS) channels with hydrophilic coating. The capillary-actuated PDMS channels direct menstrual fluid through the menstrual garment attachment without external power or pumping mechanisms. The hydrophilic coating on the PDMS channels allows menstrual fluid to flow naturally through the microfluidic system via capillary action. The PDMS material is flexible and biocompatible, making PDMS suitable for microfluidic applications in contact with biological fluids.

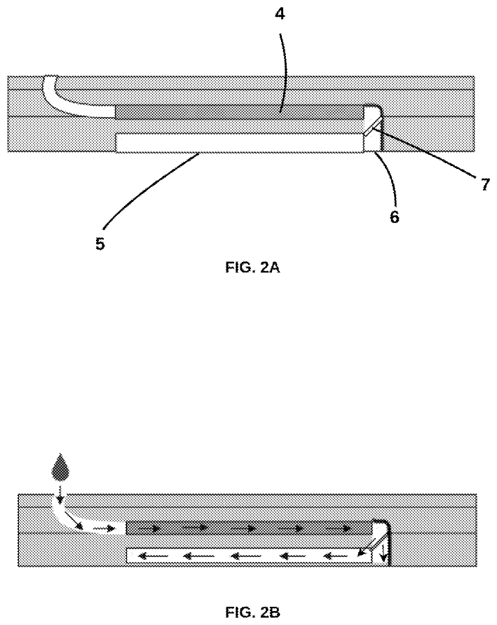

Referring to FIG. 2, a microfluidic 4 is positioned within the menstrual garment attachment. The microfluidic 4 receives menstrual fluid from the channel entrance 1 described previously with reference to FIG. 1. The microfluidic 4 comprises the capillary-actuated PDMS channels with hydrophilic coating. The microfluidic 4 directs menstrual fluid toward a lateral flow immunoassay 5 for biomarker detection. The microfluidic 4 uses capillary forces to move menstrual fluid through the system without external actuation.

With continued reference to FIG. 2, a blood reservoir system 6 is positioned within the menstrual garment attachment. The blood reservoir system 6 receives menstrual fluid from the microfluidic 4. The blood reservoir system 6 holds menstrual fluid prior to delivery to the lateral flow immunoassay 5. A polymer membrane 7 is positioned within the blood reservoir system 6. The polymer membrane 7 acts as a blood reservoir mechanism that allows a defined amount of blood for the test. The polymer membrane 7 breaks when adequate volume is reached, and menstrual fluid after that point travels down the channel and absorbs into the pad.

In an example, the pad-facing inlet comprises the channel entrance 1 described previously with reference to FIG. 1. The pad-facing inlet is positioned on the menstrual garment attachment to receive menstrual fluid from a menstrual pad or from direct contact with menstrual discharge. The pad-facing inlet directs menstrual fluid into the internal microfluidic system. The red circle 2 described previously indicates the location of the pad-facing inlet on the menstrual garment attachment. The cotton on the outside 3 described previously surrounds the pad-facing inlet and enhances absorption of menstrual fluid toward the pad-facing inlet.

In an example, the capillary-actuated PDMS channels are engineered with hydrophilic coating to allow menstrual fluid to flow naturally through the microfluidic system. The hydrophilic coating attracts menstrual fluid and promotes wetting of the channel surfaces. The hydrophilic coating reduces surface tension at the fluid-channel interface. The hydrophilic coating enables capillary action to draw menstrual fluid through the PDMS channels without external pressure or pumping. The capillary forces generated by the hydrophilic coating provide consistent fluid flow rates through the microfluidic system.

In some examples, the microfluidic channels are made from silicon wafers as an alternative material to PDMS. Silicon wafers provide a rigid substrate for microfluidic channel fabrication. Silicon wafers allow for precise microfabrication of channel geometries using established semiconductor processing techniques. Silicon wafers provide chemical resistance to biological fluids. Silicon wafers provide dimensional stability during use of the menstrual garment attachment.

In some examples, the microfluidic channels are made from glass substrates as an alternative material to PDMS. Glass substrates provide optical transparency for visualization of fluid flow through the microfluidic channels. Glass substrates provide chemical inertness to biological fluids. Glass substrates provide a smooth surface finish that promotes consistent capillary flow. Glass substrates allow for surface modification with hydrophilic coatings to enhance capillary action.

In some examples, the microfluidic channels possess a T-junction which separates the blood into feasible components for directing to the lateral flow immunoassay 5. The T-junction divides the menstrual fluid flow path into multiple branches. The T-junction directs separated components of the menstrual fluid to different regions of the lateral flow immunoassay 5. The T-junction allows for parallel processing of menstrual fluid components. The T-junction enables multi-zone detection of different biomarkers from a single menstrual fluid sample.

As shown in FIG. 2, blood enters through the microfluidic 4 and is pinched into droplets. The droplets drip onto the polymer membrane 7 that is acting as the blood reservoir mechanism. The polymer membrane 7 allows the amount of blood for the test and then the polymer membrane 7 breaks. Menstrual fluid after the polymer membrane 7 breaks travels down the channel and absorbs into the pad. The menstrual fluid then goes through the lateral flow immunoassay 5 for diagnostic testing. After 15-20 minutes, results are viewable by flipping over the pad attachment.

In an example, the internal microfluidic system is configured to process menstrual fluid without external power sources. The capillary-actuated PDMS channels with hydrophilic coating provide passive fluid transport through the microfluidic system. The passive fluid transport eliminates the need for batteries, pumps, or electronic components. The passive fluid transport reduces the cost and complexity of the menstrual garment attachment. The passive fluid transport enables use of the menstrual garment attachment in underserved communities without access to electrical infrastructure.

### Method—Volume Regulation and Membrane Rupture

In an example, the method for detecting one or more reproductive health biomarkers includes regulating fluid entry into a diagnostic zone via a hydrophilic nanoporous lipid-based membrane that ruptures at a calibrated menstrual fluid volume. The hydrophilic nanoporous lipid-based membrane is positioned between functional zones of the microfluidic system to regulate the amount of menstrual fluid that enters the diagnostic zone. The hydrophilic nanoporous lipid-based membrane is structured to break open when a defined volume of menstrual fluid is detected. The rupture of the hydrophilic nanoporous lipid-based membrane at the calibrated menstrual fluid volume enables the menstrual fluid sample to proceed to the diagnostic zone for examination through diagnostic testing.

In an example, the hydrophilic nanoporous lipid-based membrane comprises lipid nanoparticles. The lipid nanoparticles are structured to maintain membrane integrity until the calibrated menstrual fluid volume is reached. The lipid nanoparticles rupture when the menstrual fluid volume exceeds the calibrated threshold. The rupture of the lipid nanoparticles allows menstrual fluid to pass through the hydrophilic nanoporous lipid-based membrane and enter the diagnostic zone. The hydrophilic properties of the hydrophilic nanoporous lipid-based membrane attract menstrual fluid toward the membrane surface. The nanoporous structure of the hydrophilic nanoporous lipid-based membrane provides controlled permeability prior to rupture.

In some examples, the hydrophilic nanoporous lipid-based membrane ruptures between 60-120 μL of sample flow. The calibration of the hydrophilic nanoporous lipid-based membrane to rupture between 60-120 μL of sample flow ensures that a defined volume of menstrual fluid enters the diagnostic zone. The rupture threshold between 60-120 μL of sample flow provides sufficient menstrual fluid volume for accurate biomarker detection. The rupture threshold between 60-120 μL of sample flow prevents excessive menstrual fluid from overwhelming the diagnostic zone. The calibration of the rupture threshold between 60-120 μL of sample flow is achieved through selection of lipid nanoparticle composition and membrane thickness.

In some examples, the microfluidic system comprises active gating via the hydrophilic nanoporous lipid-based membrane for volume control. The active gating via the hydrophilic nanoporous lipid-based membrane provides a controlled mechanism for regulating menstrual fluid entry into the diagnostic zone. The active gating via the hydrophilic nanoporous lipid-based membrane differs from passive wicking mechanisms that do not provide volume control. The active gating via the hydrophilic nanoporous lipid-based membrane ensures that a fixed sample volume passes through to the diagnostic zone. The active gating via the hydrophilic nanoporous lipid-based membrane prevents over-absorption of menstrual fluid that would dilute the sample or interfere with biomarker detection.

In an example, the controlled mechanism provided by the hydrophilic nanoporous lipid-based membrane prevents contamination of the diagnostic zone. The hydrophilic nanoporous lipid-based membrane maintains separation between the menstrual fluid collection region and the diagnostic zone until the calibrated volume is reached. The hydrophilic nanoporous lipid-based membrane prevents premature entry of menstrual fluid into the diagnostic zone. The hydrophilic nanoporous lipid-based membrane prevents backflow of menstrual fluid from the diagnostic zone. The controlled mechanism ensures that the diagnostic zone receives menstrual fluid in a controlled manner that maintains sample integrity.

In an example, the controlled mechanism provided by the hydrophilic nanoporous lipid-based membrane ensures the test is done with an ample amount of fluid. The calibrated rupture threshold of the hydrophilic nanoporous lipid-based membrane ensures that sufficient menstrual fluid volume is collected before the menstrual fluid enters the diagnostic zone. The ample amount of fluid provided by the controlled mechanism ensures that the lateral flow immunoassay receives adequate sample volume for biomarker detection. The ample amount of fluid provided by the controlled mechanism ensures that test lines and control lines of the lateral flow immunoassay receive sufficient menstrual fluid for color development. The controlled mechanism prevents false negative results that would occur if insufficient menstrual fluid volume reached the diagnostic zone.

In some examples, the membrane layer is made from polyvinylpyrrolidone membrane as an alternative material to the lipid nanoparticle-based membrane. The polyvinylpyrrolidone membrane provides hydrophilic properties for attracting menstrual fluid. The polyvinylpyrrolidone membrane provides controlled permeability for regulating menstrual fluid entry into the diagnostic zone. The polyvinylpyrrolidone membrane is calibrated to rupture or allow fluid passage at a defined menstrual fluid volume. The polyvinylpyrrolidone membrane provides an alternative material option for volume regulation in the microfluidic system.

In some examples, the membrane layer is made from thermoplastics as an alternative material. The thermoplastics provide structural properties suitable for membrane fabrication. The thermoplastics are processed to create nanoporous structures for controlled fluid permeability. The thermoplastics are surface-treated to provide hydrophilic properties for attracting menstrual fluid. The thermoplastics provide an alternative material option that allows for different manufacturing processes and cost considerations. The thermoplastics are calibrated to provide volume control at defined menstrual fluid volumes.

In an example, the volume regulation provided by the hydrophilic nanoporous lipid-based membrane enables consistent diagnostic results across different users and menstrual flow rates. The calibrated rupture threshold ensures that the diagnostic zone receives a consistent menstrual fluid volume regardless of variations in menstrual flow rate. The consistent menstrual fluid volume provided by the volume regulation enables standardized biomarker detection. The standardized biomarker detection enables reliable interpretation of test results across different users.

### Method of Filtration and Sample Processing

In an example, the method for detecting one or more reproductive health biomarkers includes filtering the menstrual fluid via an embedded nano-lithographically treated mesh to remove tissue particulates and microbial contaminants. The embedded nano-lithographically treated mesh is positioned within the microfluidic system to intercept menstrual fluid prior to entry into the diagnostic zone. The embedded nano-lithographically treated mesh traps larger blood clots and filters tissue or debris from the menstrual fluid. The embedded nano-lithographically treated mesh removes microorganisms from the menstrual fluid to provide a clean sample for accurate analysis. The embedded nano-lithographically treated mesh ensures that the menstrual fluid entering the diagnostic components is uncontaminated.

In an example, the embedded nano-lithographically treated mesh is fabricated using nano-lithography techniques to create precise pore structures. The nano-lithography techniques allow for controlled pore sizes and pore distributions across the mesh surface. The precise pore structures of the embedded nano-lithographically treated mesh provide consistent filtration performance. The embedded nano-lithographically treated mesh removes tissue particulates that would interfere with biomarker detection. The embedded nano-lithographically treated mesh removes microbial contaminants that would affect the accuracy of the lateral flow immunoassay results.

In some examples, the filtering excludes particles above 5 μm in diameter. The exclusion of particles above 5 μm in diameter removes blood clots, tissue fragments, and cellular debris from the menstrual fluid sample. The exclusion of particles above 5 μm in diameter allows plasma and smaller blood components to pass through the embedded nano-lithographically treated mesh. The exclusion of particles above 5 μm in diameter provides a filtered sample suitable for biomarker detection by the lateral flow immunoassay. The pore-gradient design of the embedded nano-lithographically treated mesh achieves the exclusion of particles above 5 μm in diameter while maintaining adequate flow rates through the microfluidic system.

In some examples, the embedded nano-lithographically treated mesh filters pathogens through pore-gradient design. The pore-gradient design comprises pores of varying sizes arranged in a gradient pattern across the mesh. The pore-gradient design captures pathogens of different sizes at different locations within the mesh. The pore-gradient design provides microbial filtration that removes microorganisms from the menstrual fluid. The pore-gradient design ensures that the menstrual fluid sample reaching the diagnostic zone is free of microbial contaminants that would interfere with biomarker detection.

In some examples, the filter layer is made from PDMS microfilters as an alternative to the nano-lithography-treated mesh. The PDMS microfilters provide precision in fabrication due to the properties of polydimethylsiloxane material. The PDMS microfilters are biocompatible for contact with menstrual fluid. The PDMS microfilters allow plasma or whole blood to pass through while retaining larger particles. The PDMS microfilters provide an alternative filtration mechanism that achieves particle exclusion through microfabricated pore structures in the PDMS material.

In some examples, the mesh filter is incorporated using plasma bonding to the microfluidic structure. Plasma bonding creates a permanent bond between the mesh filter and the PDMS microchannels of the microfluidic system. Plasma bonding activates the surfaces of the mesh filter and the PDMS microchannels to promote adhesion. Plasma bonding provides a sealed interface between the mesh filter and the microfluidic structure that prevents menstrual fluid from bypassing the filtration step. Plasma bonding maintains the structural integrity of the filtration system during use of the menstrual garment attachment.

In some examples, the filtration segment comprises agarose beads for particle exclusion in addition to nano-mesh. The agarose beads are positioned within the filtration segment of the microfluidic system. The agarose beads provide additional particle exclusion through size-based separation. The agarose beads trap particles that pass through the nano-mesh. The combination of the agarose beads and the nano-mesh provides multi-stage filtration that removes a broader range of particle sizes from the menstrual fluid. The agarose beads are biocompatible and do not interfere with biomarker detection by the lateral flow immunoassay.

In some examples, the microfluidic system includes directional flow barriers comprising hydrophobic coating to restrict backflow. The directional flow barriers are positioned within the microfluidic channels to prevent menstrual fluid from flowing in reverse directions. The hydrophobic coating of the directional flow barriers repels menstrual fluid and prevents menstrual fluid from passing through the directional flow barriers in the reverse direction. The directional flow barriers comprising hydrophobic coating maintain unidirectional flow of menstrual fluid through the microfluidic system. The directional flow barriers comprising hydrophobic coating prevent contamination of upstream components by menstrual fluid that has passed through downstream filtration or detection zones. The directional flow barriers comprising hydrophobic coating ensure that filtered menstrual fluid does not backtrack and mix with unfiltered menstrual fluid.

In an example, the hydrophobic coating of the directional flow barriers is applied to specific regions of the microfluidic channels. The hydrophobic coating creates barriers that menstrual fluid does not cross in the reverse direction. The hydrophobic regions act as barriers to prevent fluid from moving in undesired directions while the hydrophilic regions of the microfluidic channels attract and direct menstrual fluid toward the diagnostic zone. The combination of hydrophobic directional flow barriers and hydrophilic channel surfaces provides controlled fluid routing through the microfluidic system.

In some examples, the microfluidic system includes embedded droplet pinch junctions filled with a non-reactive fluid. The embedded droplet pinch junctions are positioned within the PDMS microchannels of the microfluidic system. The embedded droplet pinch junctions segment the menstrual fluid flow into discrete droplets. The non-reactive fluid within the embedded droplet pinch junctions separates the menstrual fluid droplets and prevents mixing between droplets. The non-reactive fluid does not react with the menstrual fluid or interfere with biomarker detection. The embedded droplet pinch junctions filled with the non-reactive fluid provide controlled delivery of menstrual fluid to the diagnostic zone in discrete volumes.

In an example, the filtration provided by the embedded nano-lithographically treated mesh ensures a clean sample for analysis by the lateral flow immunoassay. The removal of tissue particulates prevents clogging of the lateral flow immunoassay components. The removal of microbial contaminants prevents false positive results from microbial interference. The filtration step prepares the menstrual fluid sample for accurate biomarker detection. The filtration step is positioned in the fluid flow path prior to the volume regulation provided by the hydrophilic nanoporous lipid-based membrane described previously.

### Method of Lateral Flow Immunoassay Processing and Biomarker Detection

In an example, the method for detecting one or more reproductive health biomarkers includes processing the filtered sample using a lateral flow immunoassay embedded in a nitrocellulose membrane. The lateral flow immunoassay embedded in the nitrocellulose membrane receives the filtered menstrual fluid from the filtration step described previously. The nitrocellulose membrane provides a porous substrate that supports the lateral flow immunoassay components. The nitrocellulose membrane has a porous structure that naturally moves fluid through capillary action. The nitrocellulose membrane moves the filtered menstrual fluid sample toward the detection zone of the lateral flow immunoassay. The lateral flow immunoassay embedded in the nitrocellulose membrane provides biomarker detection without external power sources or laboratory equipment.

In an example, the lateral flow immunoassay includes reagents reactive to disease biomarkers. The reagents reactive to disease biomarkers are positioned within the lateral flow immunoassay at defined locations along the nitrocellulose membrane. The reagents reactive to disease biomarkers comprise antibodies or other binding molecules configured to bind to target biomarkers present in the filtered menstrual fluid sample. The reagents reactive to disease biomarkers produce detectable signals when target biomarkers bind to the reagents. The detectable signals comprise color changes visible at test lines and control lines of the lateral flow immunoassay. The reagents reactive to disease biomarkers enable detection of reproductive health conditions through analysis of menstrual fluid.

In some examples, the lateral flow immunoassay comprises a capillary-driven immunochromatographic assay. The capillary-driven immunochromatographic assay utilizes capillary forces to transport the filtered menstrual fluid sample through the nitrocellulose membrane. The capillary-driven immunochromatographic assay does not require external pumping or pressure to move the filtered menstrual fluid sample. The capillary-driven immunochromatographic assay relies on the porous structure of the nitrocellulose membrane to generate capillary action. The capillary-driven immunochromatographic assay provides passive fluid transport that enables biomarker detection without electronic components. The capillary-driven immunochromatographic assay provides an alternative terminology for describing the lateral flow immunoassay detection mechanism.

In some examples, the lateral flow immunoassay includes multi-zone detection configured to detect one or more analytes selected from the group consisting of HPV-16 E6 oncoproteins, estradiol, IL-6, and fibroid-associated hormones. The multi-zone detection comprises multiple test lines positioned at different locations along the nitrocellulose membrane. Each test line of the multi-zone detection contains immobilized capture molecules specific to a different target analyte. The multi-zone detection allows simultaneous detection of multiple biomarkers from a single filtered menstrual fluid sample. The multi-zone detection configured to detect HPV-16 E6 oncoproteins includes reagents that bind to HPV-16 E6 oncoproteins associated with cervical cancer. The multi-zone detection configured to detect estradiol includes reagents that bind to estradiol hormone present in menstrual fluid. The multi-zone detection configured to detect IL-6 includes reagents that bind to interleukin-6 inflammatory marker. The multi-zone detection configured to detect fibroid-associated hormones includes reagents that bind to hormones associated with uterine fibroids.

In some examples, the one or more reproductive health biomarkers are associated with conditions selected from the group consisting of cervical cancer, endometriosis, breast cancer, uterine fibroids, and pelvic inflammation. The lateral flow immunoassay detects biomarkers associated with cervical cancer through reagents reactive to HPV-related oncoproteins and other cervical cancer indicators. The lateral flow immunoassay detects biomarkers associated with endometriosis through reagents reactive to inflammatory markers and hormonal indicators present in menstrual fluid of individuals with endometriosis. The lateral flow immunoassay detects biomarkers associated with breast cancer through reagents reactive to breast cancer-associated proteins present in menstrual fluid. The lateral flow immunoassay detects biomarkers associated with uterine fibroids through reagents reactive to fibroid-associated hormones and proteins. The lateral flow immunoassay detects biomarkers associated with pelvic inflammation through reagents reactive to inflammatory markers indicative of pelvic inflammatory conditions.

In some examples, the lateral flow immunoassay detects sexually transmitted infections as target analytes. The lateral flow immunoassay includes reagents reactive to sexually transmitted infection pathogens or pathogen-associated markers present in menstrual fluid. The detection of sexually transmitted infections through menstrual fluid analysis provides a non-invasive screening method. The lateral flow immunoassay configured to detect sexually transmitted infections includes test lines with immobilized capture molecules specific to sexually transmitted infection biomarkers.

In some examples, the lateral flow immunoassay detects ovarian cancer biomarkers as target analytes. The lateral flow immunoassay includes reagents reactive to ovarian cancer-associated proteins or markers present in menstrual fluid. The detection of ovarian cancer biomarkers through menstrual fluid analysis provides early screening capability. The lateral flow immunoassay configured to detect ovarian cancer biomarkers includes test lines with immobilized capture molecules specific to ovarian cancer indicators.

In some examples, the one or more reproductive health biomarkers comprise myeloperoxidase (MPO) as an inflammatory biomarker. Myeloperoxidase is an enzyme released by neutrophils during inflammatory responses. The lateral flow immunoassay includes reagents reactive to myeloperoxidase for detection of inflammatory activity. The detection of myeloperoxidase in menstrual fluid provides indication of inflammatory conditions within the reproductive system. Myeloperoxidase serves as a wellness indicator for inflammatory activity within the menstrual environment. The detection of myeloperoxidase as a wellness indicator for inflammatory activity within the menstrual environment differs from cardiovascular risk applications of myeloperoxidase detection. Cardiovascular risk applications of myeloperoxidase detection focus on systemic myeloperoxidase levels as indicators of cardiovascular disease risk. The menstrual garment attachment detects myeloperoxidase specifically within the menstrual environment to assess inflammatory activity related to reproductive health conditions rather than cardiovascular risk. The lateral flow immunoassay configured to detect myeloperoxidase includes test lines with immobilized capture molecules specific to myeloperoxidase protein.

In an example, the lateral flow immunoassay measures myeloperoxidase protein levels rather than enzymatic activity of myeloperoxidase. The measurement of myeloperoxidase protein levels via the lateral flow immunoassay counts active, inhibited, and modified forms of myeloperoxidase. The measurement of myeloperoxidase protein levels improves standardization in heterogeneous menstrual blood compared to enzymatic activity measurements. The lateral flow immunoassay provides consistent myeloperoxidase detection across menstrual fluid samples with varying compositions.

In an example, the detection of myeloperoxidase as an inflammatory biomarker enables longitudinal tracking and personalized trend analysis. The menstrual garment attachment provides myeloperoxidase measurements over multiple menstrual cycles. The longitudinal tracking of myeloperoxidase levels allows users to monitor changes in inflammatory activity within the menstrual environment over time. The personalized trend analysis compares current myeloperoxidase levels to previous myeloperoxidase levels from the same user. The personalized trend analysis differs from absolute diagnostic thresholds that compare myeloperoxidase levels to population-based reference ranges. The personalized trend analysis accounts for individual variation in baseline myeloperoxidase levels.

In an example, the reagents reactive to disease biomarkers are conjugated to colored nanoparticles as described previously. The colored nanoparticles produce visible color changes at test lines when target biomarkers bind to the reagents. The colored nanoparticles are visible through the translucent polymer layer described previously. The color changes at test lines indicate the presence of specific disease biomarkers in the filtered menstrual fluid sample. The color changes at control lines indicate proper function of the lateral flow immunoassay.

In an example, the lateral flow immunoassay embedded in the nitrocellulose membrane provides results based on the composition of the filtered menstrual fluid sample. The lateral flow immunoassay detects biomarkers that react with the reagents reactive to disease biomarkers. The lateral flow immunoassay allows for detection of infections, hormonal imbalances, and disease conditions through analysis of menstrual fluid. The lateral flow immunoassay provides rapid results without laboratory processing. The lateral flow immunoassay enables at-home testing for reproductive health conditions.

### Method—Visible Results and Trend Analysis

In an example, the method for detecting one or more reproductive health biomarkers includes producing a visible result on an exterior of the menstrual garment attachment for biomarker detection. The visible result is produced at the results region of the lateral flow immunoassay described previously. The visible result comprises color changes at test lines and control lines of the lateral flow immunoassay. The visible result is positioned on the exterior of the menstrual garment attachment such that users observe the visible result without disassembling the menstrual garment attachment. The visible result provides indication of biomarker presence or absence based on the color changes produced by the lateral flow immunoassay.

In an example, the visible result is produced through accumulation of colored nanoparticles at test lines and control lines of the lateral flow immunoassay. The colored nanoparticles migrate through the nitrocellulose membrane with the filtered menstrual fluid sample. The colored nanoparticles accumulate at test lines when biomarker-antibody complexes bind to immobilized capture molecules. The colored nanoparticles accumulate at control lines when labeled antibodies bind to immobilized molecules regardless of biomarker presence. The accumulation of colored nanoparticles produces visible color changes that constitute the visible result.

In some examples, the visible result comprises colorimetric nanoparticles visible through a transparent window embedded in the menstrual garment attachment. The transparent window is positioned over the results region of the lateral flow immunoassay. The transparent window comprises the translucent polymer layer described previously. The transparent window allows light transmission for visualization of the colorimetric nanoparticles. The colorimetric nanoparticles produce color changes that are visible through the transparent window. The transparent window protects the results region from contamination while allowing users to observe the visible result. The colorimetric nanoparticles visible through the transparent window provide clear indication of test results without requiring removal of protective coverings.

In an example, the colorimetric nanoparticles produce distinct color changes at test lines that indicate biomarker presence. The colorimetric nanoparticles produce distinct color changes at control lines that indicate proper function of the lateral flow immunoassay. The color changes produced by the colorimetric nanoparticles are distinguishable from the background color of the nitrocellulose membrane. The color changes produced by the colorimetric nanoparticles are visible under ambient lighting conditions. The colorimetric nanoparticles provide visual indication of test results without electronic readers or specialized equipment.

In some examples, the visible result is available within 10-30 minutes of menstrual fluid contact with the inlet. The menstrual fluid contacts the inlet at the pad-facing inlet described previously. The menstrual fluid flows through the microfluidic system, the volume regulation membrane, and the filtration components before reaching the lateral flow immunoassay. The lateral flow immunoassay processes the filtered menstrual fluid sample and produces the visible result. The time period of 10-30 minutes from menstrual fluid contact with the inlet to availability of the visible result allows for complete processing of the menstrual fluid sample through the microfluidic system and the lateral flow immunoassay. The visible result is stable and readable after the 10-30 minute processing period.

In some examples, the diagnostic result is produced within 15 minutes visible on the exterior of the pad. The 15 minute time period provides rapid results for users. The diagnostic result produced within 15 minutes allows users to obtain biomarker detection information during a single use session. The diagnostic result produced within 15 minutes eliminates the need for extended waiting periods. The diagnostic result produced within 15 minutes is visible on the exterior of the menstrual garment attachment through the transparent window described previously. The 15 minute time period for producing the diagnostic result is achieved through optimization of the microfluidic flow rates, the volume regulation membrane rupture timing, and the lateral flow immunoassay reaction kinetics.

In an example, the visible result remains stable on the exterior of the menstrual garment attachment after the initial processing period. The visible result does not fade or change after the color development is complete. The stable visible result allows users to interpret the test results at a convenient time after the processing period. The stable visible result allows users to document or photograph the test results for record keeping. The stable visible result provides a permanent record of the biomarker detection for the single use of the menstrual garment attachment.

In some examples, the method includes comparing the visible result to one or more previous results to establish personalized trend analysis. The personalized trend analysis tracks changes in biomarker levels over multiple menstrual cycles. The personalized trend analysis compares the visible result from a current use of the menstrual garment attachment to visible results from previous uses of the menstrual garment attachment. The personalized trend analysis identifies patterns in biomarker presence or intensity over time. The personalized trend analysis provides information about changes in reproductive health status based on longitudinal biomarker data.

In an example, the personalized trend analysis differs from reliance on absolute diagnostic thresholds. Absolute diagnostic thresholds compare a single test result to population-based reference ranges. Absolute diagnostic thresholds do not account for individual variation in baseline biomarker levels. The personalized trend analysis accounts for individual variation by comparing current results to previous results from the same user. The personalized trend analysis identifies changes from an individual's baseline rather than deviations from population averages. The personalized trend analysis provides individualized health monitoring that is responsive to each user's unique biomarker profile.

In an example, the personalized trend analysis enables longitudinal tracking of reproductive health biomarkers. Longitudinal tracking comprises collection of biomarker data over multiple time points. Longitudinal tracking identifies trends in biomarker levels that indicate changes in health status. Longitudinal tracking detects gradual changes in biomarker levels that would not be apparent from a single test result. Longitudinal tracking provides early indication of developing health conditions through identification of biomarker trends. Longitudinal tracking enables users to monitor reproductive health over extended time periods.

In an example, the personalized trend analysis is performed by comparing the intensity of color changes at test lines across multiple uses of the menstrual garment attachment. The intensity of color changes at test lines correlates with the concentration of target biomarkers in the menstrual fluid sample. Comparison of color change intensity across multiple uses indicates whether biomarker concentrations are increasing, decreasing, or remaining stable over time. The comparison of color change intensity provides quantitative trend information from qualitative visual results.

In an example, users document the visible result from each use of the menstrual garment attachment for personalized trend analysis. Users photograph the visible result using a mobile device. Users record the date and menstrual cycle day associated with each visible result. Users compare photographs of visible results from multiple uses to identify trends in biomarker presence or intensity. The documentation of visible results enables personalized trend analysis without electronic data storage within the menstrual garment attachment.

In some examples, the personalized trend analysis is facilitated by the QR-linked visual guide described previously. The QR-linked visual guide provides instructions for documenting and comparing visible results over time. The QR-linked visual guide provides reference images for interpreting color change intensity. The QR-linked visual guide provides guidance for identifying trends in biomarker levels based on comparison of visible results from multiple uses.

In an example, the personalized trend analysis provides health monitoring capability for users in underserved communities. The personalized trend analysis does not require laboratory infrastructure for longitudinal biomarker tracking. The personalized trend analysis does not require electronic health record systems. The personalized trend analysis enables users to monitor reproductive health trends using sequential visible results from the menstrual garment attachment. The personalized trend analysis provides accessible health monitoring for populations without access to regular medical care or laboratory testing.

In an example, a diagnostic pad attachment apparatus for detecting disease biomarkers in menstrual fluid comprises a non-powered mechanical design optimized for temperature neutrality. The non-powered mechanical design does not include batteries, electronic circuits, or powered components. The non-powered mechanical design relies on passive fluid transport mechanisms including capillary action through the microfluidic channels and the lateral flow immunoassay. The non-powered mechanical design eliminates the need for external power sources during operation of the diagnostic pad attachment apparatus. The non-powered mechanical design enables use of the diagnostic pad attachment apparatus in locations without access to electrical infrastructure.

In an example, the non-powered mechanical design is optimized for temperature neutrality. The temperature neutrality of the non-powered mechanical design allows the diagnostic pad attachment apparatus to function across a range of ambient temperatures without temperature control systems. The temperature neutrality of the non-powered mechanical design eliminates the need for heating elements, cooling elements, or temperature sensors. The temperature neutrality of the non-powered mechanical design allows the diagnostic pad attachment apparatus to operate at body temperature when worn by a user. The temperature neutrality of the non-powered mechanical design allows the diagnostic pad attachment apparatus to be stored at ambient temperatures without refrigeration. The temperature neutrality of the non-powered mechanical design allows the diagnostic pad attachment apparatus to be transported and distributed without cold chain logistics.

In an example, the reagents of the lateral flow immunoassay are selected and formulated for stability across a range of temperatures. The reagents maintain reactivity to target biomarkers at temperatures encountered during storage, transport, and use of the diagnostic pad attachment apparatus. The reagents do not require refrigeration for stability. The reagents do not require temperature-controlled storage conditions. The formulation of the reagents for temperature stability enables distribution of the diagnostic pad attachment apparatus in regions without reliable refrigeration infrastructure.

In an example, the materials of the diagnostic pad attachment apparatus are selected for stability across a range of temperatures. The polydimethylsiloxane (PDMS) microchannels maintain structural integrity and hydrophilic coating functionality across a range of temperatures. The nitrocellulose membrane maintains porosity and capillary flow properties across a range of temperatures. The hydrophilic nanoporous lipid-based membrane maintains calibrated rupture threshold across a range of temperatures. The translucent polymer layer maintains optical clarity and seal integrity across a range of temperatures. The selection of temperature-stable materials enables consistent performance of the diagnostic pad attachment apparatus in varying environmental conditions.

In some examples, the diagnostic pad attachment apparatus comprises a non-powered mechanical design optimized for low-cost mass production. The non-powered mechanical design reduces manufacturing costs by eliminating electronic components, batteries, and powered systems. The non-powered mechanical design reduces material costs by utilizing passive fluid control mechanisms rather than active pumping systems. The non-powered mechanical design reduces assembly complexity by eliminating electrical connections and circuit board integration. The non-powered mechanical design enables high-volume manufacturing using established fabrication techniques for microfluidic devices and lateral flow immunoassays.

In some examples, the optimization for low-cost mass production includes selection of materials that are available at low cost and in large quantities. The polydimethylsiloxane (PDMS) material for the microchannels is available at low cost for high-volume manufacturing. The nitrocellulose membrane material is available at low cost for high-volume manufacturing. The textile materials for the surface layer are available at low cost for high-volume manufacturing. The selection of low-cost materials reduces the per-unit cost of the diagnostic pad attachment apparatus.

In some examples, the optimization for low-cost mass production includes design features that simplify manufacturing processes. The multi-layer housing of the diagnostic pad attachment apparatus is configured for assembly using automated manufacturing equipment. The bonding of the translucent polymer layer to the surface layer and to the lateral flow immunoassay is achieved using manufacturing processes suitable for high-volume production. The integration of the microfluidic system, the filtration components, and the lateral flow immunoassay is achieved through manufacturing processes that minimize manual assembly steps. The simplification of manufacturing processes reduces labor costs and increases production throughput.

In some examples, the optimization for low-cost mass production includes design features that reduce material waste during manufacturing. The dimensions of the diagnostic pad attachment apparatus are selected to maximize the number of units produced from standard material sheet sizes. The cutting and forming processes for the diagnostic pad attachment apparatus components are designed to minimize scrap material. The reduction of material waste during manufacturing reduces the per-unit cost of the diagnostic pad attachment apparatus.

In an example, the non-powered mechanical design optimized for fluid control enables consistent diagnostic performance without electronic control systems. The capillary-actuated PDMS channels with hydrophilic coating provide consistent fluid flow rates through passive capillary action. The hydrophilic nanoporous lipid-based membrane provides consistent volume regulation through calibrated rupture threshold. The embedded nano-lithographically treated mesh provides consistent filtration through passive particle exclusion. The lateral flow immunoassay provides consistent biomarker detection through passive capillary-driven immunochromatographic reaction. The passive fluid control mechanisms provide consistent diagnostic performance across units produced through mass production.

In an example, the non-powered mechanical design abolishes the need for electronic components and power sources. The abolishment of electronic components eliminates the cost of electronic circuit boards, sensors, and microprocessors. The abolishment of electronic components eliminates the cost of batteries or other power sources. The abolishment of electronic components eliminates the complexity of electronic assembly and testing during manufacturing. The abolishment of electronic components eliminates the risk of electronic component failure during use. The abolishment of electronic components eliminates the need for electronic waste disposal after use.

In an example, the abolishment of electronic components and power sources increases accessibility in resource-constrained settings. Resource-constrained settings include underserved communities without reliable access to electrical infrastructure. Resource-constrained settings include regions without access to battery supplies or battery disposal facilities. Resource-constrained settings include locations where electronic medical devices are not available or affordable. The diagnostic pad attachment apparatus provides diagnostic capability in resource-constrained settings through the non-powered mechanical design. The diagnostic pad attachment apparatus does not require electricity, batteries, or electronic infrastructure for operation. The diagnostic pad attachment apparatus provides accessible diagnostic testing for populations in resource-constrained settings.

In an example, the non-powered mechanical design increases accessibility by reducing the cost of the diagnostic pad attachment apparatus. The reduced cost enables distribution of the diagnostic pad attachment apparatus to populations with limited financial resources. The reduced cost enables public health programs to provide the diagnostic pad attachment apparatus at low cost or no cost to users in underserved communities. The reduced cost enables wider distribution of the diagnostic pad attachment apparatus compared to electronic diagnostic devices with higher manufacturing costs.

In an example, the non-powered mechanical design increases accessibility by simplifying use of the diagnostic pad attachment apparatus. The diagnostic pad attachment apparatus does not require users to operate electronic controls or interfaces. The diagnostic pad attachment apparatus does not require users to charge batteries or connect to power sources. The diagnostic pad attachment apparatus does not require users to interpret electronic displays or digital readouts. The diagnostic pad attachment apparatus provides visible results through colorimetric changes that are interpreted without electronic assistance. The simplification of use enables users without technical training to operate the diagnostic pad attachment apparatus.

In an example, the non-powered mechanical design increases accessibility by enabling distribution through existing supply chains. The diagnostic pad attachment apparatus does not require specialized storage conditions for electronic components. The diagnostic pad attachment apparatus does not require specialized handling for batteries or power sources. The diagnostic pad attachment apparatus is distributed through supply chains used for conventional menstrual hygiene products. The distribution through existing supply chains enables the diagnostic pad attachment apparatus to reach populations in remote or underserved areas.

In an example, a top view of a menstrual garment attachment for detecting disease biomarkers in menstrual fluid shows a channel entrance positioned on the menstrual garment attachment. The channel entrance provides an inlet for menstrual fluid to flow into the internal microfluidic system of the menstrual garment attachment. The channel entrance is positioned at a location on the menstrual garment attachment that corresponds to the area where menstrual fluid is collected during use. The channel entrance receives menstrual fluid from the surface layer of the menstrual garment attachment. The channel entrance directs menstrual fluid into the microfluidic channels for processing and biomarker detection.

In an example, a red circle is positioned on the menstrual garment attachment to indicate the location of the channel entrance. The red circle provides a visual indicator that identifies the channel entrance location for users. The red circle is visible on the top surface of the menstrual garment attachment. The red circle assists users in positioning the menstrual garment attachment correctly during application. The red circle indicates the area where menstrual fluid enters the diagnostic system of the menstrual garment attachment. The red circle provides a reference point for proper orientation of the menstrual garment attachment relative to the user's body.

In an example, cotton is positioned on the outside of the menstrual garment attachment. The cotton on the outside comprises the surface layer of the menstrual garment attachment. The cotton on the outside is configured to receive menstrual fluid upon contact with the user's body. The cotton on the outside enhances absorption of menstrual fluid. The cotton on the outside quickly draws and retains menstrual fluid. The cotton on the outside absorbs large quantities of fluid and helps maintain dryness on the surface of the menstrual garment attachment. The cotton on the outside is breathable and allows for constant air circulation while maintaining absorbency. The cotton on the outside comprises small pores for menstrual fluid to pass through to the microfluidic channels.

In some examples, the cotton on the outside is replaced with bamboo fibers. Bamboo fibers have natural antibacterial properties. Bamboo fibers provide a sustainable alternative to cotton. Bamboo fibers quickly draw and retain fluid while providing antibacterial protection. Bamboo fibers are breathable and allow for constant air circulation. Bamboo fibers comprise small pores for menstrual fluid to pass through to the microfluidic channels.

In an example, the top of the menstrual garment attachment is coated with hydrophobic and hydrophilic coatings to ensure flow to the correct area. The hydrophilic coatings are positioned in regions of the top surface that correspond to the fluid flow path toward the channel entrance. The hydrophilic coatings attract menstrual fluid and promote wetting of the coated surfaces. The hydrophilic coatings direct menstrual fluid toward the channel entrance through capillary action. The hydrophilic coatings reduce surface tension at the fluid-surface interface to facilitate fluid movement toward the channel entrance.

In an example, the hydrophobic coatings are positioned in regions of the top surface that surround the hydrophilic regions. The hydrophobic coatings repel menstrual fluid and prevent menstrual fluid from spreading to undesired areas of the menstrual garment attachment. The hydrophobic coatings act as barriers that confine menstrual fluid to the hydrophilic regions. The hydrophobic coatings prevent menstrual fluid from moving in undesired directions away from the channel entrance. The hydrophobic coatings maintain the directed flow of menstrual fluid toward the channel entrance.

In an example, the combination of hydrophobic and hydrophilic coatings creates a patterned surface that controls menstrual fluid flow on the top of the menstrual garment attachment. The patterned surface directs menstrual fluid from the point of contact with the surface layer toward the channel entrance. The patterned surface prevents menstrual fluid from spreading laterally across the top surface of the menstrual garment attachment. The patterned surface ensures that menstrual fluid enters the channel entrance rather than being absorbed into peripheral regions of the menstrual garment attachment. The patterned surface provides consistent fluid routing regardless of the exact location where menstrual fluid contacts the surface layer.

In an example, the hydrophilic coatings comprise surface treatments applied to the cotton or bamboo fiber material of the surface layer. The surface treatments modify the surface energy of the fiber material to increase hydrophilicity. The surface treatments are applied in defined patterns that correspond to the desired fluid flow paths. The surface treatments are durable and maintain hydrophilic properties throughout the use period of the menstrual garment attachment.

In an example, the hydrophobic coatings comprise surface treatments applied to the cotton or bamboo fiber material of the surface layer. The surface treatments modify the surface energy of the fiber material to increase hydrophobicity. The surface treatments are applied in defined patterns that correspond to the barrier regions surrounding the fluid flow paths. The surface treatments are durable and maintain hydrophobic properties throughout the use period of the menstrual garment attachment.

In an example, the hydrophilic coatings extend from the surface layer through to the channel entrance. The hydrophilic coatings provide a continuous hydrophilic pathway from the point of menstrual fluid contact to the channel entrance. The continuous hydrophilic pathway ensures uninterrupted capillary flow of menstrual fluid toward the channel entrance. The continuous hydrophilic pathway prevents menstrual fluid from being trapped or pooling at interfaces between different regions of the menstrual garment attachment.

In an example, the hydrophobic coatings extend around the perimeter of the channel entrance. The hydrophobic coatings around the perimeter of the channel entrance prevent menstrual fluid from bypassing the channel entrance. The hydrophobic coatings around the perimeter of the channel entrance direct menstrual fluid into the channel entrance rather than around the channel entrance. The hydrophobic coatings around the perimeter of the channel entrance ensure that menstrual fluid entering the surface layer is routed through the channel entrance into the microfluidic system.

In an example, the top view of the menstrual garment attachment shows the spatial relationship between the channel entrance, the red circle indicator, and the cotton on the outside. The channel entrance is positioned within the area indicated by the red circle. The cotton on the outside surrounds the channel entrance and the red circle. The hydrophilic coatings are positioned on the cotton on the outside in the region surrounding and leading to the channel entrance. The hydrophobic coatings are positioned on the cotton on the outside in the regions peripheral to the hydrophilic coated regions.

In an example, the top view of the menstrual garment attachment shows the overall shape and dimensions of the menstrual garment attachment. The menstrual garment attachment has a shape configured to fit within a menstrual pad or underwear. The menstrual garment attachment has dimensions that allow the menstrual garment attachment to be positioned in the area where menstrual fluid is discharged during use. The channel entrance is positioned at a location on the menstrual garment attachment that corresponds to the expected location of menstrual fluid discharge when the menstrual garment attachment is worn.

In an example, a pad attachment technology for detecting disease biomarkers in menstrual fluid comprises three main parts: a channel, a microfluidic system, and a blood reservoir system. The channel receives menstrual fluid from the surface layer of the pad attachment. The channel directs menstrual fluid toward the microfluidic system for processing. The microfluidic system receives menstrual fluid from the channel and processes the menstrual fluid for biomarker detection. The blood reservoir system receives processed menstrual fluid from the microfluidic system and regulates the volume of menstrual fluid that proceeds to the diagnostic components.

In an example, the channel comprises an inlet portion that receives menstrual fluid from the surface layer of the pad attachment. The channel extends from the inlet portion toward the microfluidic system. The channel is configured to direct menstrual fluid flow in a defined direction from the inlet portion to the microfluidic system. The channel comprises hydrophilic surfaces that promote capillary flow of menstrual fluid through the channel. The channel comprises hydrophobic barriers that prevent menstrual fluid from flowing in undesired directions.

In an example, the microfluidic system comprises capillary-actuated channels that receive menstrual fluid from the channel. The microfluidic system comprises polydimethylsiloxane (PDMS) channels with hydrophilic coating. The PDMS channels are flexible and biocompatible for contact with menstrual fluid. The hydrophilic coating on the PDMS channels allows menstrual fluid to flow naturally through the microfluidic system via capillary action. The microfluidic system directs menstrual fluid toward the blood reservoir system and the lateral flow immunoassay for biomarker detection.

In an example, the blood reservoir system comprises a polymer membrane that regulates the volume of menstrual fluid that proceeds to the lateral flow immunoassay. The polymer membrane is positioned within the blood reservoir system between the microfluidic system and the lateral flow immunoassay. The polymer membrane acts as a blood reservoir mechanism that allows a defined amount of menstrual fluid for the diagnostic test. The polymer membrane is structured to maintain integrity until a calibrated volume of menstrual fluid is reached. The polymer membrane breaks when the adequate volume of menstrual fluid is met.

In an example, the lateral flow immunoassay is positioned downstream from the blood reservoir system in the fluid flow path. The lateral flow immunoassay receives menstrual fluid that has passed through the polymer membrane after the polymer membrane breaks. The lateral flow immunoassay comprises a nitrocellulose membrane that supports biomarker detection components. The lateral flow immunoassay comprises reagents reactive to disease biomarkers present in menstrual fluid. The lateral flow immunoassay produces visible color changes at test lines and control lines to indicate biomarker presence or absence.

In an example, menstrual fluid enters the pad attachment through the channel. The menstrual fluid flows from the channel into the microfluidic system. The microfluidic system processes the menstrual fluid and directs the menstrual fluid toward the blood reservoir system. The menstrual fluid enters the microfluidic system and is pinched into droplets. The droplets are formed through the geometry of the microfluidic channels. The droplets provide controlled volumes of menstrual fluid for processing.

In an example, the droplets drip onto the polymer membrane that is acting as the blood reservoir mechanism. The polymer membrane receives the droplets from the microfluidic system. The polymer membrane accumulates menstrual fluid from the droplets. The polymer membrane maintains integrity while menstrual fluid accumulates on the polymer membrane. The polymer membrane allows the amount of menstrual fluid for the diagnostic test to accumulate before the polymer membrane breaks.

In an example, the polymer membrane breaks when the adequate volume of menstrual fluid is met. The breaking of the polymer membrane occurs when the accumulated menstrual fluid volume exceeds the calibrated threshold of the polymer membrane. The breaking of the polymer membrane releases the accumulated menstrual fluid to proceed to the lateral flow immunoassay. Menstrual fluid that arrives after the polymer membrane breaks travels down the channel and absorbs into the pad. The absorption of excess menstrual fluid into the pad prevents overflow of menstrual fluid into the diagnostic components.

In an example, the menstrual fluid that is released when the polymer membrane breaks proceeds to the lateral flow immunoassay for diagnostic testing. The menstrual fluid flows through the lateral flow immunoassay via capillary action. The menstrual fluid contacts the reagents reactive to disease biomarkers within the lateral flow immunoassay. The reagents reactive to disease biomarkers bind to target biomarkers present in the menstrual fluid. The binding of target biomarkers to the reagents produces visible color changes at test lines of the lateral flow immunoassay.

In an example, the lateral flow immunoassay produces visible color changes at control lines to indicate proper function of the lateral flow immunoassay. The control lines receive labeled antibodies from the lateral flow immunoassay regardless of biomarker presence in the menstrual fluid. The visible color changes at the control lines confirm that the menstrual fluid has flowed through the lateral flow immunoassay and that the reagents are functioning properly. The visible color changes at the test lines and the control lines constitute the diagnostic result of the lateral flow immunoassay.

In an example, results of the diagnostic test are viewable after 15-20 minutes by flipping over the pad attachment. The 15-20 minute time period allows for complete processing of the menstrual fluid through the microfluidic system, the blood reservoir system, and the lateral flow immunoassay. The 15-20 minute time period allows for complete color development at the test lines and the control lines of the lateral flow immunoassay. The flipping over of the pad attachment exposes the results region of the lateral flow immunoassay for user observation. The results region is positioned on the pad attachment such that the results region is visible when the pad attachment is flipped over.

In an example, the user observes the visible color changes at the test lines and the control lines after flipping over the pad attachment. The user interprets the visible color changes to determine the presence or absence of target biomarkers in the menstrual fluid sample. The presence of visible color changes at the test lines indicates the presence of target biomarkers associated with disease conditions. The absence of visible color changes at the test lines indicates the absence of target biomarkers in the menstrual fluid sample. The presence of visible color changes at the control lines indicates that the lateral flow immunoassay has functioned properly.

In an example, the three main parts of the pad attachment technology work in sequence to process menstrual fluid and produce diagnostic results. The channel receives menstrual fluid and directs the menstrual fluid to the microfluidic system. The microfluidic system processes the menstrual fluid and forms droplets that drip onto the polymer membrane. The blood reservoir system accumulates menstrual fluid until the polymer membrane breaks at the calibrated volume. The lateral flow immunoassay receives the released menstrual fluid and produces visible color changes for biomarker detection. The sequential operation of the channel, the microfluidic system, and the blood reservoir system provides controlled delivery of menstrual fluid to the lateral flow immunoassay for accurate diagnostic results.

In an example, the polymer membrane comprises lipid nanoparticles that are structured to rupture at the calibrated menstrual fluid volume. The lipid nanoparticles maintain membrane integrity until the calibrated volume is reached. The lipid nanoparticles rupture when the menstrual fluid volume exceeds the calibrated threshold. The rupture of the lipid nanoparticles allows menstrual fluid to pass through the polymer membrane and enter the lateral flow immunoassay. The calibration of the lipid nanoparticles to rupture at a defined volume ensures that a consistent volume of menstrual fluid reaches the lateral flow immunoassay for biomarker detection.

In some examples, the polymer membrane is calibrated to rupture between 60-120 μL of sample flow. The calibration of the polymer membrane to rupture between 60-120 μL of sample flow ensures that sufficient menstrual fluid volume is collected before the menstrual fluid enters the lateral flow immunoassay. The rupture threshold between 60-120 μL of sample flow provides adequate menstrual fluid volume for accurate biomarker detection by the lateral flow immunoassay. The rupture threshold between 60-120 μL of sample flow prevents excessive menstrual fluid from overwhelming the lateral flow immunoassay.

In an example, the microfluidic system comprises a T-junction that separates the menstrual fluid into components for directing to the lateral flow immunoassay. The T-junction divides the menstrual fluid flow path into multiple branches. The T-junction directs separated components of the menstrual fluid to different regions of the lateral flow immunoassay. The T-junction allows for parallel processing of menstrual fluid components. The T-junction enables multi-zone detection of different biomarkers from a single menstrual fluid sample.

In an example, the microfluidic system comprises filtration components that remove tissue particulates and microbial contaminants from the menstrual fluid before the menstrual fluid reaches the lateral flow immunoassay. The filtration components are positioned within the microfluidic system upstream from the blood reservoir system. The filtration components trap larger blood clots and filter tissue or debris from the menstrual fluid. The filtration components ensure that the menstrual fluid entering the lateral flow immunoassay is uncontaminated for accurate biomarker detection.

In an example, the pad attachment is configured for single use and disposal after the diagnostic test is complete. The pad attachment does not require sanitization between uses. The single use configuration eliminates infection risk associated with improper sanitization of reusable devices. The single use configuration provides a hygienic diagnostic solution for users. The disposal of the pad attachment after single use eliminates the need for cleaning or maintenance of the diagnostic components.

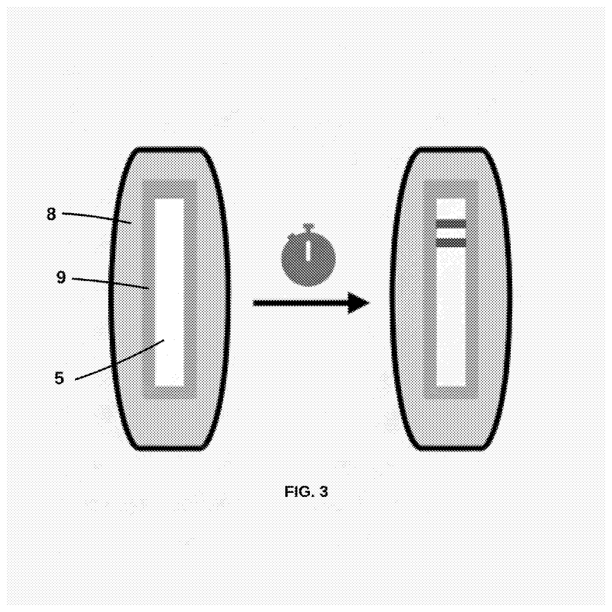

Referring to FIG. 3, a transparent polymer 9 is positioned over the lateral flow immunoassay 5 to cover and prevent contamination of the diagnostic apparatus while allowing the lateral flow immunoassay 5 reading to be visible. The transparent polymer 9 provides a protective barrier between the external environment and the lateral flow immunoassay 5. The transparent polymer 9 prevents dust, debris, moisture, and other external contaminants from contacting the lateral flow immunoassay 5. The transparent polymer 9 maintains the integrity of the lateral flow immunoassay 5 during handling and use of the menstrual garment attachment.

In an example, the transparent polymer 9 is positioned over a first portion of the lateral flow immunoassay 5. The transparent polymer 9 exposes a second portion of the lateral flow immunoassay 5. The first portion of the lateral flow immunoassay 5 that is covered by the transparent polymer 9 includes components that are susceptible to contamination. The second portion of the lateral flow immunoassay 5 that is exposed includes the results region where test lines and control lines appear. The positioning of the transparent polymer 9 over the first portion and exposing the second portion allows users to view test results without compromising the diagnostic accuracy of the lateral flow immunoassay 5.

In an example, the transparent polymer 9 transmits light to allow users to observe color changes at the test lines and control lines of the lateral flow immunoassay 5. The transparent polymer 9 does not distort or obscure the color changes produced by colorimetric nanoparticles within the lateral flow immunoassay 5. The transparent polymer 9 provides optical clarity for visualization of the lateral flow immunoassay 5 results. The transparent polymer 9 allows users to interpret the diagnostic results by observing the visible color changes through the transparent polymer 9.

In an example, the transparent polymer 9 comprises a hydrophobic polymer material. The hydrophobic polymer material repels water and aqueous fluids including menstrual fluid. The hydrophobic polymer material prevents menstrual fluid from adhering to the exterior surface of the transparent polymer 9. The hydrophobic polymer material maintains visibility of the lateral flow immunoassay 5 results by preventing fluid accumulation on the viewing surface. The hydrophobic polymer material prevents moisture from penetrating through the transparent polymer 9 to the lateral flow immunoassay 5.

In some examples, the transparent polymer 9 comprises polyisoprene. Polyisoprene is a synthetic rubber material with hydrophobic properties. Polyisoprene provides flexibility to the transparent polymer 9. Polyisoprene provides durability to the transparent polymer 9. Polyisoprene maintains optical clarity for visualization of the lateral flow immunoassay 5 results. Polyisoprene is biocompatible for contact with skin and menstrual fluid. Polyisoprene provides resistance to degradation from exposure to menstrual fluid.

In some examples, the transparent polymer 9 comprises materials other than polyisoprene. In some examples, the transparent polymer 9 comprises silicone. In some examples, the transparent polymer 9 comprises polyethylene. In some examples, the transparent polymer 9 comprises polypropylene. The selection of the transparent polymer 9 material is based on the desired optical clarity, flexibility, durability, and biocompatibility for the menstrual garment attachment application.

With continued reference to FIG. 3, an adhesive 8 is positioned on the menstrual garment attachment to enable attachment to a menstrual pad. The adhesive 8 is positioned on a back surface of the menstrual garment attachment opposite the surface where the transparent polymer 9 is positioned. The adhesive 8 provides a bonding mechanism between the menstrual garment attachment and the menstrual pad. The adhesive 8 secures the menstrual garment attachment in position on the menstrual pad during use.

In an example, the adhesive 8 comprises a pressure-sensitive adhesive. The pressure-sensitive adhesive forms a bond when pressure is applied to the menstrual garment attachment against the menstrual pad. The pressure-sensitive adhesive does not require heat, water, or solvent activation to form the bond. The pressure-sensitive adhesive provides immediate adhesion when the menstrual garment attachment is pressed onto the menstrual pad. The pressure-sensitive adhesive maintains the bond between the menstrual garment attachment and the menstrual pad throughout the use period.

In an example, the adhesive 8 is configured to adhere to the textile surface of a menstrual pad. The adhesive 8 bonds to the fibers of the menstrual pad surface layer. The adhesive 8 maintains adhesion to the menstrual pad surface during user movement. The adhesive 8 maintains adhesion to the menstrual pad surface when exposed to moisture from menstrual fluid. The adhesive 8 prevents the menstrual garment attachment from shifting or detaching from the menstrual pad during use.

In an example, the adhesive 8 is positioned on the menstrual garment attachment such that the adhesive 8 does not interfere with the fluid flow path through the menstrual garment attachment. The adhesive 8 is positioned around the perimeter of the menstrual garment attachment or in regions that do not overlap with the channel entrance 1 described previously with reference to FIG. 1. The positioning of the adhesive 8 ensures that menstrual fluid enters the channel entrance 1 and flows through the microfluidic 4 without obstruction from the adhesive 8.

In an example, the adhesive 8 is covered by a protective backing prior to use of the menstrual garment attachment. The protective backing prevents the adhesive 8 from bonding to surfaces during storage and handling. The protective backing is removed by the user prior to application of the menstrual garment attachment to the menstrual pad. The removal of the protective backing exposes the adhesive 8 for bonding to the menstrual pad surface.

In an example, the adhesive 8 comprises a silicone-based adhesive. The silicone-based adhesive provides adhesion to textile surfaces of menstrual pads. The silicone-based adhesive is optimized for attachment to the textile top-sheet of feminine hygiene products. The silicone-based adhesive is optimized for overnight wear. The silicone-based adhesive is optimized for moisture resistance. The silicone-based adhesive maintains adhesion when exposed to menstrual fluid and body moisture during extended use periods.

In an example, the adhesive 8 is configured to allow removal of the menstrual garment attachment from the menstrual pad after use. The adhesive 8 provides sufficient adhesion to maintain the position of the menstrual garment attachment during use. The adhesive 8 allows the menstrual garment attachment to be peeled away from the menstrual pad without damaging the menstrual pad. The removability of the menstrual garment attachment allows users to dispose of the menstrual garment attachment separately from the menstrual pad if desired.

As shown in FIG. 3, the transparent polymer 9 and the adhesive 8 are positioned on opposite sides of the menstrual garment attachment. The transparent polymer 9 is positioned on the top side of the menstrual garment attachment facing away from the menstrual pad. The adhesive 8 is positioned on the bottom side of the menstrual garment attachment facing toward the menstrual pad. The positioning of the transparent polymer 9 on the top side allows users to view the lateral flow immunoassay 5 results through the transparent polymer 9. The positioning of the adhesive 8 on the bottom side allows the menstrual garment attachment to be secured to the menstrual pad.

In an example, the lateral flow immunoassay 5 is positioned between the transparent polymer 9 and the adhesive 8 within the menstrual garment attachment. The lateral flow immunoassay 5 is protected from above by the transparent polymer 9. The lateral flow immunoassay 5 is supported from below by the structure of the menstrual garment attachment that includes the adhesive 8. The positioning of the lateral flow immunoassay 5 between the transparent polymer 9 and the adhesive 8 provides a protected environment for biomarker detection.

In an example, the transparent polymer 9 is bonded to the menstrual garment attachment to form a seal around the lateral flow immunoassay 5. The seal formed by the bonding of the transparent polymer 9 prevents external contaminants from entering the region containing the lateral flow immunoassay 5. The seal formed by the bonding of the transparent polymer 9 prevents menstrual fluid from leaking out of the menstrual garment attachment through the top surface. The seal formed by the bonding of the transparent polymer 9 maintains the sterility of the lateral flow immunoassay 5 components.

In an example, the transparent polymer 9 provides a viewing window for observing the diagnostic results of the lateral flow immunoassay 5. The viewing window is positioned over the results region of the lateral flow immunoassay 5 where test lines and control lines appear. The viewing window allows users to observe the presence or absence of color changes at the test lines and control lines. The viewing window provides clear visualization of the diagnostic results without requiring removal of the transparent polymer 9.

In an example, the combination of the transparent polymer 9 and the adhesive 8 enables the menstrual garment attachment to function as a removable diagnostic accessory. The adhesive 8 allows the menstrual garment attachment to be attached to existing menstrual pads. The transparent polymer 9 allows users to view diagnostic results while the menstrual garment attachment remains attached to the menstrual pad. The combination of the transparent polymer 9 and the adhesive 8 provides a self-contained diagnostic device that is applied to and removed from menstrual pads without integration into the pad structure.

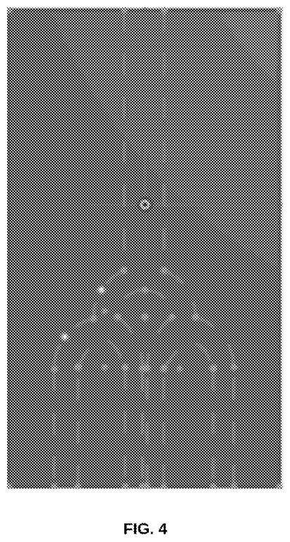

Referring to FIG. 4, a microfluidic design that allows for capillary flow is shown. The microfluidic design comprises a network of channels configured to direct menstrual fluid through the menstrual garment attachment using capillary forces. The microfluidic design enables passive fluid transport without external pumping mechanisms or power sources. The microfluidic design utilizes the surface properties of the channel materials to generate capillary action that draws menstrual fluid through the system.

In an example, the microfluidic design comprises capillary-actuated channels that receive menstrual fluid from the inlet of the menstrual garment attachment. The capillary-actuated channels are dimensioned to generate capillary forces that draw menstrual fluid into and through the channels. The dimensions of the capillary-actuated channels are selected to provide consistent capillary flow rates for menstrual fluid processing. The capillary-actuated channels have cross-sectional dimensions that promote capillary action based on the surface tension properties of menstrual fluid.

In an example, the capillary-actuated channels comprise polydimethylsiloxane (PDMS) material. The PDMS material is flexible and conforms to the shape of the menstrual garment attachment during use. The PDMS material is biocompatible for contact with menstrual fluid. The PDMS material provides a substrate that is amenable to surface modification for controlling fluid flow properties. The PDMS material allows for fabrication of microchannels with precise dimensions using established microfabrication techniques.

In an example, the capillary-actuated channels comprise hydrophilic coating on the channel surfaces. The hydrophilic coating is applied to the interior surfaces of the PDMS channels. The hydrophilic coating attracts menstrual fluid and promotes wetting of the channel surfaces. The hydrophilic coating reduces the contact angle between menstrual fluid and the channel surfaces. The reduced contact angle generated by the hydrophilic coating enhances capillary action within the channels. The hydrophilic coating enables menstrual fluid to flow naturally through the microfluidic system via capillary forces without external pressure or pumping.

In an example, the microfluidic design comprises hydrophilic regions that define the fluid flow paths through the menstrual garment attachment. The hydrophilic regions are positioned along the intended fluid flow paths from the inlet toward the diagnostic components. The hydrophilic regions attract menstrual fluid and direct menstrual fluid along the defined flow paths. The hydrophilic regions provide continuous wetting surfaces that maintain capillary flow of menstrual fluid through the microfluidic system.

In an example, the microfluidic design comprises hydrophobic regions that confine menstrual fluid to the intended flow paths. The hydrophobic regions are positioned adjacent to the hydrophilic regions. The hydrophobic regions repel menstrual fluid and prevent menstrual fluid from spreading beyond the defined flow paths. The hydrophobic regions act as barriers that contain menstrual fluid within the capillary-actuated channels. The hydrophobic regions prevent menstrual fluid from flowing in undesired directions within the microfluidic system.

In an example, the combination of hydrophilic regions and hydrophobic regions creates a patterned surface within the microfluidic design that controls menstrual fluid routing. The patterned surface directs menstrual fluid from the inlet toward the filtration components, the volume regulation membrane, and the lateral flow immunoassay. The patterned surface prevents menstrual fluid from bypassing the intended processing steps. The patterned surface ensures that menstrual fluid follows the defined flow path through the microfluidic system for proper sample processing and biomarker detection.

In an example, the hydrophilic regions of the microfluidic design are created through surface treatment of the PDMS channel surfaces. The surface treatment modifies the surface energy of the PDMS material to increase hydrophilicity. The surface treatment comprises plasma treatment, chemical coating, or other surface modification techniques that render the PDMS surfaces hydrophilic. The surface treatment is applied in defined patterns that correspond to the intended fluid flow paths within the microfluidic design.

In an example, the hydrophobic regions of the microfluidic design comprise untreated PDMS surfaces or surfaces treated to increase hydrophobicity. The untreated PDMS surfaces have inherent hydrophobic properties that repel aqueous fluids including menstrual fluid. The hydrophobic treatment comprises application of hydrophobic coatings or surface modifications that increase the contact angle between menstrual fluid and the treated surfaces. The hydrophobic regions are positioned in defined patterns that correspond to the barrier regions surrounding the fluid flow paths.

In an example, the microfluidic design comprises directional flow barriers that restrict backflow of menstrual fluid within the capillary-actuated channels. The directional flow barriers comprise hydrophobic coating lines positioned at locations within the microfluidic channels. The hydrophobic coating lines create barriers that menstrual fluid does not cross in the reverse direction. The directional flow barriers maintain unidirectional flow of menstrual fluid through the microfluidic system from the inlet toward the diagnostic components.

In an example, the directional flow barriers prevent contamination of upstream components by menstrual fluid that has passed through downstream processing steps. The directional flow barriers prevent filtered menstrual fluid from backtracking and mixing with unfiltered menstrual fluid. The directional flow barriers prevent menstrual fluid that has contacted the lateral flow immunoassay reagents from flowing back toward the inlet. The directional flow barriers ensure that each processing step within the microfluidic system receives menstrual fluid in the intended sequence.

In an example, the microfluidic design comprises channel geometries that enhance capillary flow. The channel geometries include channel width variations that modulate capillary pressure along the flow path. The channel geometries include channel depth variations that control flow velocity through different regions of the microfluidic system. The channel geometries include corner features that promote capillary wicking of menstrual fluid along the channel edges.

In an example, the microfluidic design comprises a narrow-neck geometry that provides passive flow resistance tuning. The narrow-neck geometry comprises a constricted region within the capillary-actuated channels. The constricted region has a reduced cross-sectional area compared to adjacent channel regions. The narrow-neck geometry creates increased flow resistance at the constricted region. The increased flow resistance at the narrow-neck geometry regulates the flow rate of menstrual fluid through the microfluidic system. The narrow-neck geometry provides passive regulation of menstrual fluid flow without active valves or electronic control systems.

In an example, the microfluidic design comprises channel junctions that divide menstrual fluid flow into multiple branches. The channel junctions include T-junctions that separate menstrual fluid into two flow paths. The channel junctions include Y-junctions that divide menstrual fluid flow at defined angles. The channel junctions direct separated portions of menstrual fluid to different regions of the diagnostic components. The channel junctions enable parallel processing of menstrual fluid for multi-zone biomarker detection.

In an example, the microfluidic design comprises inlet normalization features that account for variations in menstrual fluid composition. The inlet normalization features condition the menstrual fluid as the menstrual fluid enters the capillary-actuated channels. The inlet normalization features provide consistent fluid properties for downstream processing regardless of variations in the composition of menstrual fluid from different users or different menstrual cycle phases. The inlet normalization features differ from differential chromatographic separation techniques by providing inline conditioning of the menstrual fluid sample.

In an example, the capillary flow generated by the microfluidic design provides consistent fluid transport rates across different use conditions. The capillary flow is generated by the surface properties of the hydrophilic coated channels rather than by external pressure or pumping. The capillary flow rate is determined by the channel dimensions, the surface energy of the hydrophilic coating, and the surface tension of the menstrual fluid. The capillary flow provides reproducible fluid transport that enables consistent biomarker detection by the lateral flow immunoassay.

In an example, the microfluidic design is configured to operate at body temperature when the menstrual garment attachment is worn by a user. The capillary flow properties of the microfluidic design are maintained across the temperature range encountered during use. The hydrophilic coating maintains wetting properties at body temperature. The hydrophobic regions maintain barrier properties at body temperature. The temperature stability of the microfluidic design enables consistent fluid routing and processing during use of the menstrual garment attachment.

In an example, the microfluidic design is fabricated using microfabrication techniques that allow for precise control of channel dimensions and surface properties. The microfabrication techniques include soft lithography for creating PDMS channel structures. The microfabrication techniques include plasma treatment for creating hydrophilic surface regions. The microfabrication techniques include selective coating application for creating hydrophobic barrier regions. The microfabrication techniques enable reproducible manufacturing of the microfluidic design for consistent performance across multiple menstrual garment attachment units.

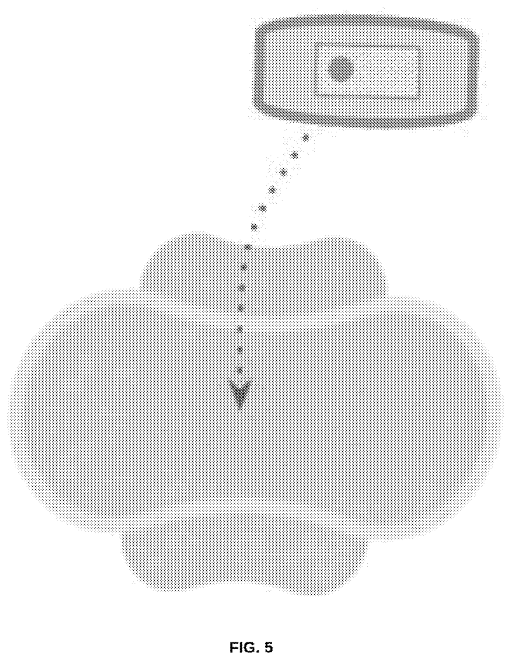

Referring to FIG. 5, the attachment adheres to a menstrual garment through an adhesive positioned on the back of the attachment. The attachment is configured as a standalone diagnostic accessory that is applied to existing menstrual products. The attachment does not form a structural component of the menstrual garment. The attachment is removably attached to the menstrual garment through the adhesive on the back of the attachment.

In an example, the adhesive on the back of the attachment causes the attachment to stick to the menstrual garment. The adhesive is positioned on a surface of the attachment that faces toward the menstrual garment when the attachment is applied. The adhesive contacts the surface of the menstrual garment when the attachment is pressed onto the menstrual garment. The adhesive forms a bond between the attachment and the menstrual garment that maintains the position of the attachment during use.

In an example, the attachment is applied to the menstrual garment by a user prior to wearing the menstrual garment. The user removes a protective backing from the adhesive on the back of the attachment. The removal of the protective backing exposes the adhesive surface. The user positions the attachment on the menstrual garment at a location corresponding to where menstrual fluid is collected during use. The user presses the attachment onto the menstrual garment to engage the adhesive with the surface of the menstrual garment.

In an example, the adhesive on the back of the attachment is configured to adhere to the textile surface of the menstrual garment. The menstrual garment comprises a sanitary pad with a textile top-sheet. The adhesive bonds to the fibers of the textile top-sheet when the attachment is pressed onto the menstrual garment. The adhesive maintains adhesion to the textile surface during user movement. The adhesive maintains adhesion to the textile surface when exposed to moisture from menstrual fluid.

In an example, the adhesive on the back of the attachment comprises a pressure-sensitive adhesive. The pressure-sensitive adhesive forms a bond when pressure is applied to the attachment against the menstrual garment. The pressure-sensitive adhesive does not require heat, water, or solvent activation to form the bond. The pressure-sensitive adhesive provides immediate adhesion when the attachment is pressed onto the menstrual garment.

In an example, the adhesive on the back of the attachment comprises a silicone-based adhesive. The silicone-based adhesive provides adhesion to textile surfaces of menstrual garments. The silicone-based adhesive is optimized for attachment to the textile top-sheet of feminine hygiene products. The silicone-based adhesive is optimized for overnight wear during extended use periods. The silicone-based adhesive is optimized for moisture resistance when exposed to menstrual fluid and body moisture.

In an example, the attachment maintains position on the menstrual garment throughout the use period. The adhesive on the back of the attachment prevents the attachment from shifting during user movement. The adhesive on the back of the attachment prevents the attachment from detaching from the menstrual garment during use. The attachment remains in contact with menstrual fluid collected by the menstrual garment throughout the use period.

In an example, the attachment is removable from the menstrual garment after use. The adhesive on the back of the attachment provides sufficient adhesion to maintain position during use. The adhesive on the back of the attachment allows the attachment to be peeled away from the menstrual garment without damaging the menstrual garment. The user grasps an edge of the attachment and pulls the attachment away from the menstrual garment to remove the attachment. The removal of the attachment separates the adhesive bond between the attachment and the menstrual garment.

In an example, the attachment is disposed of after single use. The attachment is removed from the menstrual garment after the diagnostic test is complete. The attachment is discarded separately from the menstrual garment or together with the menstrual garment. The single use configuration eliminates the need for sanitization of the attachment between uses.

With continued reference to FIG. 5, the attachment is positioned on the menstrual garment at a location that corresponds to the area where menstrual fluid is discharged during use. The positioning of the attachment on the menstrual garment places the inlet of the attachment in contact with menstrual fluid collected by the menstrual garment. The positioning of the attachment on the menstrual garment allows menstrual fluid to enter the microfluidic system of the attachment for processing and biomarker detection.

In an example, the attachment is positioned on the menstrual garment with the adhesive surface facing toward the menstrual garment and the diagnostic components facing away from the menstrual garment. The orientation of the attachment positions the inlet of the attachment in contact with the surface of the menstrual garment where menstrual fluid is absorbed. The orientation of the attachment positions the translucent polymer layer and the results region of the lateral flow immunoassay facing away from the menstrual garment for user observation.

In an example, the attachment is configured to function independently of the menstrual garment structure. The attachment does not require integration into the layers of the menstrual garment. The attachment does not require the menstrual garment to have specialized features for receiving the attachment. The attachment is applied to conventional menstrual garments including disposable sanitary pads and reusable menstrual pads. The attachment provides diagnostic capability to existing menstrual products through the adhesive attachment mechanism.

In an example, the adhesive on the back of the attachment is positioned such that the adhesive does not obstruct the fluid flow path through the attachment. The adhesive is positioned around the perimeter of the attachment or in regions that do not overlap with the inlet of the attachment. The positioning of the adhesive ensures that menstrual fluid enters the inlet and flows through the microfluidic system without obstruction from the adhesive.

In an example, the attachment is applied to the menstrual garment without tools or specialized equipment. The user applies the attachment by hand using the adhesive on the back of the attachment. The application of the attachment does not require medical training or technical expertise. The application of the attachment is performed by the user at home without assistance from healthcare providers.

In an example, the attachment is applied to the menstrual garment prior to the menstrual garment being worn by the user. The user applies the attachment to the menstrual garment while the menstrual garment is positioned on a flat surface. The user then wears the menstrual garment with the attachment applied. The attachment collects and processes menstrual fluid during the use period while the menstrual garment is worn.

In an example, the attachment is applied to the menstrual garment after the menstrual garment is positioned in underwear. The user positions the menstrual garment in underwear. The user then applies the attachment to the menstrual garment while the menstrual garment is positioned in the underwear. The application of the attachment to the menstrual garment after positioning in underwear allows the user to align the attachment with the expected location of menstrual fluid discharge.

In an example, the standalone diagnostic accessory configuration of the attachment enables use with a variety of menstrual garment types. The attachment is applied to thin sanitary pads. The attachment is applied to thick sanitary pads. The attachment is applied to overnight sanitary pads. The attachment is applied to reusable cloth menstrual pads. The adhesive on the back of the attachment provides adhesion to the textile surfaces of different menstrual garment types.

In an example, the standalone diagnostic accessory configuration of the attachment enables retrofitting of existing menstrual products with diagnostic capability. Users purchase the attachment separately from menstrual garments. Users apply the attachment to menstrual garments that the users already own or purchase. The attachment provides diagnostic capability without requiring users to purchase specialized diagnostic menstrual garments. The attachment enables users to add diagnostic functionality to preferred menstrual garment brands and styles.

The numerical values, percentages, and ranges disclosed herein are not to be understood as being strictly limited to the exact numerical values recited. Instead, unless otherwise specified, each such value is intended to mean both the recited value and a functionally equivalent range surrounding that value. For example, a concentration disclosed as “10 nM” is intended to mean “about 10 nM.”

### Fluid Transport System Embodiments

In an example, the fluid transport system of the menstrual garment attachment comprises one or more of: a laminar flow membrane; a porous wicking membrane; a depth filtration layer; a hydrophobic-patterned flow channel; a stacked membrane assembly; a volume-metering zone; a flow-stop barrier; an overflow bypass channel; a passive capillary pump; a microfluidic chip comprising etched or molded microchannels; or combinations thereof. The fluid transport system is configured to deliver menstrual fluid to the detection module without requiring external power.

In an example, the fluid transport system comprises a laminar flow membrane configured to direct menstrual fluid in a substantially unidirectional flow path toward the detection module. The laminar flow membrane provides controlled fluid transport that minimizes turbulence and ensures consistent sample delivery to the detection module.

In an example, the fluid transport system comprises a stacked membrane architecture comprising two or more membrane layers having different porosities arranged to progressively filter and meter menstrual fluid as it advances toward the detection module. The stacked membrane architecture provides both filtration and volume control functions within a single integrated structure.

In an example, the fluid transport system comprises a microfluidic chip comprising one or more etched or molded microchannels. The microfluidic chip may be fabricated from polydimethylsiloxane (PDMS), silicon, glass, or thermoplastic materials. The etched or molded microchannels provide precise control over fluid flow paths and volumes.

In an example, the fluid transport system comprises a porous wicking membrane that draws menstrual fluid through the device via capillary action. The porous wicking membrane may comprise cellulose, glass fiber, or synthetic polymer materials configured to provide consistent wicking rates.

In an example, the fluid transport system comprises a depth filtration layer configured to remove cellular debris, tissue fragments, and particulate matter from the menstrual fluid as it passes through the device. The depth filtration layer may comprise multiple layers of fibrous material with progressively smaller pore sizes.

In an example, the fluid transport system comprises a hydrophobic-patterned flow channel that directs menstrual fluid along defined pathways. The hydrophobic patterning creates barriers that prevent fluid from spreading laterally and ensure directed flow toward the detection module.

In an example, the fluid transport system comprises a volume-metering zone configured to deliver a defined volume of menstrual fluid to the detection module. The volume-metering zone may comprise a chamber or channel segment with a known volume that fills before releasing fluid to downstream components.

In an example, the fluid transport system comprises a flow-stop barrier configured to halt fluid flow at a defined location within the device. The flow-stop barrier may comprise a hydrophobic region, a physical barrier, or a dissolvable membrane that controls the timing of fluid delivery to the detection module.

In an example, the fluid transport system comprises an overflow bypass channel configured to divert excess menstrual fluid away from the detection module. The overflow bypass channel prevents flooding of the detection zone and ensures that only a controlled volume of fluid reaches the detection module.

In an example, the fluid transport system comprises a passive capillary pump configured to draw menstrual fluid through the device without external power. The passive capillary pump may comprise a high-surface-area absorbent material positioned downstream of the detection module that creates sustained capillary pressure to drive fluid flow.

A number of implementations have been described. Nevertheless, it will be understood that various modifications may be made without departing from the spirit and scope of the disclosure. Accordingly, other implementations are within the scope of the following claims.

### Optical Detection and Biomarker Embodiments

In an example, the detection module comprises an optical detection system. The optical detection system may comprise colorimetric detection, fluorescence detection, chemiluminescence detection, or combinations thereof. The optical detection system is configured to generate a detectable optical signal in response to the presence or concentration of one or more target analytes in the menstrual fluid.

In an example, the one or more target analytes comprise one or more biomarkers selected from the group consisting of biomarkers associated with inflammation, hormonal status, iron status, metabolic status, and reproductive health. Biomarkers associated with inflammation may include myeloperoxidase (MPO), C-reactive protein (CRP), interleukin-6 (IL-6), tumor necrosis factor-alpha (TNF-α), and other inflammatory cytokines. Biomarkers associated with hormonal status may include estradiol, progesterone, luteinizing hormone (LH), follicle-stimulating hormone (FSH), and other reproductive hormones. Biomarkers associated with iron status may include ferritin, transferrin, and hemoglobin. Biomarkers associated with metabolic status may include glucose, lactate, and other metabolites. Biomarkers associated with reproductive health may include oncoproteins, pathogen-associated antigens, and hormone receptor binding proteins.

In an example, the mobile device application is configured to establish a within-individual baseline for one or more target analytes and generate cycle-specific contextual output relative to the individual's prior cycle data. The within-individual baseline is established by collecting and storing biomarker data from multiple menstrual cycles for the same user. The cycle-specific contextual output compares current biomarker levels to the user's historical baseline rather than population averages, enabling personalized health monitoring.

In an example, the method for detecting one or more reproductive health biomarkers further comprises normalizing a detected analyte signal against a reference signal comprising hemoglobin concentration, total protein concentration, or a combination thereof, to generate a within-individual normalized biomarker value. The normalization accounts for variations in menstrual fluid composition between cycles and between individuals, improving the accuracy and reproducibility of biomarker measurements.

1. A menstrual garment attachment for tracking reproductive health biomarkers in menstrual fluid, the menstrual garment attachment comprising:
a housing configured to be attachable to an absorbent menstrual article;
a detection module disposed within the housing and configured to detect one or more target analytes in the menstrual fluid and produce a user-readable output;
a fluid transport system comprising one or more fluid pathways configured to passive convey menstrual fluid from a garment-facing surface of the housing to the detection module, wherein the menstrual fluid is passively conveyed by capillary action, wicking, absorption, gravity-assisted flow, or combinations thereof;
a fluid inlet positioned on the garment-facing surface of the housing and configured to receive menstrual fluid from the absorbent article; and
a cover layer positioned over at least a first portion of the detection module and exposing a second portion of the detection module.

2. The menstrual garment attachment of claim 1, wherein the detection module comprises a lateral flow immunoassay comprising a porous membrane.

3. The menstrual garment attachment of claim 1, wherein the detection module comprises one or more electrochemical sensors configured to measure an electrical signal correlated with the concentration of one or more target analytes.

4. The menstrual garment attachment of claim 1, further comprising a near-field communication (NFC) chip configured to transmit analytical data from the detection module to a mobile device application.

5. The menstrual garment attachment of claim 4, wherein the mobile device application is configured to store and aggregate detection data across a plurality of menstrual cycles for longitudinal tracking, establish a within-individual baseline for one or more target analytes, and generate cycle-specific contextual output relative to the individual's prior cycle data.

6. The menstrual garment attachment of claim 1, wherein the translucent polymer layer comprises a hydrophobic, optically transparent, biocompatible polymer.

7. The menstrual garment attachment of claim 6, wherein the hydrophobic polymer is selected from the group consisting of polyisoprene, silicone, polydimethylsiloxane (PDMS), polyethylene, polypropylene, thermoplastic polyurethane (TPU), cyclic olefin copolymer (COC), and cyclic olefin polymer (COP).

8. The menstrual garment attachment of claim 1, wherein the cover layer is bonded to the housing and to the detection module to form a fluid-tight seal.

9. The menstrual garment attachment of claim 2, wherein the lateral flow immunoassay comprises a collection pad positioned at a first end of the lateral flow immunoassay.

10. The menstrual garment attachment of claim 9, wherein the lateral flow immunoassay further comprises a sample pad and a fluid-conditioning reagent disposed within a capillary conduit.

11. The menstrual garment attachment of claim 10, wherein the lateral flow immunoassay further comprises:
a conjugate pad;
a results region comprising one or more test lines and one or more control lines arranged to perform a sandwich immunoassay, competitive inhibition assay, or multiplex detection of two or more target analytes via spatially separated test zones;
and a wick pad.

12. The menstrual garment attachment of claim 1, further comprising wings extending from opposite sides of the menstrual garment attachment.

13. The menstrual garment attachment of claim 1, wherein the housing comprises an antimicrobial material comprising charcoal bamboo fiber fabric.

14. The menstrual garment attachment of claim 1, further comprising a surface-mountable label, wherein the surface-mountable label comprises pictographic instructions, a QR code, or a combination thereof.

15. The menstrual garment attachment of claim 1, wherein the fluid transport system comprises one or more of:
a laminar flow membrane, a porous wicking membrane, a depth filtration layer, a hydrophobic-patterned flow channel, a stacked membrane assembly, a volume-metering zone, a flow-stop barrier, an overflow bypass channel, a passive capillary pump, and a microfluidic chip comprising etched or molded microchannels;
wherein the fluid transport system is configured to deliver menstrual fluid to the detection module.

16. The menstrual garment attachment of claim 15, wherein the fluid transport system comprises a laminar flow membrane configured to direct menstrual fluid in a unidirectional flow path toward the detection module.

17. The menstrual garment attachment of claim 15, wherein the fluid transport system comprises two or more membrane layers having different porosities arranged to progressively filter and meter menstrual fluid as the menstrual fluid advances toward the detection module.

18. The menstrual garment attachment of claim 15, wherein the fluid transport system comprises a microfluidic chip comprising one or more etched or molded microchannels.

19. The menstrual garment attachment of claim 1, wherein the detection module comprises an optical detection system.

20. A system for tracking reproductive health biomarkers in menstrual fluid, the system comprising:
a menstrual garment attachment comprising a housing configured to be attachable to a menstrual absorbent article, a detection module disposed within the housing and configured to detect one or more target analytes in the menstrual fluid and produce a user-readable output, and a fluid transport system configured to passively convey menstrual fluid from a garment-facing surface of the housing to the detection module;
a wireless communication module integrated into the menstrual garment attachment, wherein the wireless communication module comprises a near-field communication (NFC) chip and/or a Bluetooth low energy (BLE) transmitter; and
a mobile device application configured to receive, display, store, and analyze transmitted results over time for longitudinal trend analysis.
